(19) United States
(12) Patent Application Publication
(10) Pub. No.: US 2022/0053769 A1
(43) Pub. Date: Feb. 24, 2022

(54) MICROBACTERIUM ESTERAROMATICUM STRAIN, COMPOSITION COMPRISING THE SAME, AND USES THEREOF

(71) Applicant: FUTURECO BIOSCIENCE, S.A., OLÈRDOLA (ES)

(72) Inventors: José Manuel LARA SÁNCHEZ, OLÈRDOLA (ES); Carolina FERNÁNDEZ CASTILLO, OLÈRDOLA (ES)

(21) Appl. No.: 17/312,460

(22) PCT Filed: Dec. 17, 2019
(86) PCT No.: PCT/EP2019/085774

§ 371 Date: Jun. 10, 2021

(30) Foreign Application Priority Data
Dec. 18, 2018 (EP) 18382937.3

(51) Int. Cl. A01N 63/20; C12N 1/20

(52) U.S. Cl. CPC A01N 63/20; C12N 1/205

(57) ABSTRACT

The present invention relates to a new isolated strain of the microorganism Microbacterium esteraromaticum which has nematicidal effect, the bacterial culture and the composition comprising it and their uses in the control of nematode infection in a plant. Also refers to a method to obtain the mutant strain of the invention.

This application claims the benefit of European Patent Application EP18382937.3 filed on Dec. 18, 2018.

### TECHNICAL FIELD

The present invention relates to the field of phytosanitary products. It provides a new isolated strain of the microorganism Microbacterium esteraromaticum, compositions comprising it and their use in the biological control of diseases caused by parasitic nematodes in plants.

### BACKGROUND ART

Parasitic nematodes cause important plant losses of great economic importance around the world, being among the major limiting factors in field and plantation crop production. Parasitic nematodes include root-knot nematodes, e.g., the Meloidogyne genus; cyst nematodes, e.g., the Globodera genus; and root-lesion nematodes, e.g., the Pratylenchus genus.

The most commonly used nematode management strategy is chemical control but, although is effective, it may lead to soil pollution problems, affecting biodiversity and having a negative impact on human health. Over the last decades, researchers have tried to develop non-chemical and ecofriendly approaches. Some studies have evaluated the potential of plant extracts comprising rhizobacteria strains to inhibit parasitic nematodes, wherein it has been suggested that the nematicidal effect is also performed by biocidal substances released by the plant. In Sturz A V et al. (Sturz A V and Kimpinski J 2004 Plan and Soil 262:241-249) rhizobacterial antagonists of plant parasitic nematodes were identified in marigold extracts but their efficacy was restricted to the nematodes present in the soil and they were not effective against the parasitic nematodes within the root tissues.

It is important to point out that the environmental and host conditions affect the biological activity of the bacteria strains, making their effectiveness generally variable and significantly lower than the reference chemical products.

In spite of the efforts made, there is a need for bacterial strains with nematicidal properties which overcome all or part of the limitations shown by the bacterial strains already known in the state of the art in controlling parasitic plant nematodes, specially being effective against the parasitic nematodes present in the root tissues.

### SUMMARY OF THE INVENTION

The inventors have surprisingly found one strain of the Microbacterium esteraromaticum species (hereinafter also referred as “bacterial strain B24” or “B24”) which presents antagonist activity against plant-parasitic nematodes, such as root-knot nematodes and cyst nematodes, which performs its activity in the nematodes present in the plant (i.e.: root) and that exercises said activity in a wide range of soil pH.

As it is shown below, the strain of the invention has been tested in growth chamber and greenhouse trials and has demonstrated to be efficient, in controlling eggs and juvenile root-knot nematodes (RKNs) Meloidogyne incognita and Meloidogyne javanica and against the potato cyst nematodes (PCNs) Globodera rostochiensis and Globodera pallida.

It is remarkable that the in vitro efficacy of the strain of the invention against the tested nematodes, in terms of egg hatching inhibition, was similar to the one achieved using the reference nematicidal chemical in RKN nematodes (reference chemical was Fenamiphos) (FIGS. 1 and 2). Surprisingly, the nematicidal effect of the strain of the invention in vitro against cysts of PCN was better than that of the reference chemical (Oxamyl) (example 5, FIGS. 7 and 8). This anti-nematode effect, in terms of nematode control, was also detected in vivo, measuring the final population of RKN eggs per plant and per gram of fresh root (RKN reproduction). This in vivo effect was achieved using the bacteria of the invention in several forms: as pellet (example 6.1), as Technical Grade Active Ingredient (TGAI) (example 6.2) and as formulated prototype (oil dispersion) (example 6.3). Surprisingly in cucumber plants in vivo the efficacy on final population and on reproduction was better than the reference chemical (see example 6.4).

From these experimental data it could be concluded that the bacterial strain of the present invention has a surprising effect controlling a broad range of nematodes, independently of their developmental stage and part of the plant. This is the first time that it is reported a strain of M. esteraromaticum with such particular effect.

In addition, to such remarkable anti-nematode activity profile, the inventors also found that the strain of the present invention was safe, as it is shown in example 7 when mice were fed with the strain of the invention as TGAI no pathogenicity was found.

Without being bound to any theory, the inventors believe that such nematicidal effect is due to the fact that the strain of the present invention contacts the host and releases certain enzymes (see example 1, wherein nematicidal effect is observed with the supernatant) and participates in the Induced Systemic Resistance (ISR) of the plant.

Furthermore, the strain of the invention has the ability to colonize and survive in soils with pH ranging from 5 to 10.

The pH range of soils, can range from ultra-acidic, which are soils that have pH lower than 3.5, to very strongly alkaline soils, which have pH higher than 9. The soil pH has a clear impact on plant nutrients availability. This is the reason why there are some plant species that can grow in some soils, and other that cannot, depending on their growth requirements. On the other hand, it is well-recognized by the skilled person in the art that environment conditions such as pH can negatively affect the viability/activity of a particular microorganism. The inventors have surprisingly found that the strain of the invention can survive at extreme pH values, between 5 and 10. This is a further valuable advantage because it means that the strain of the invention can exert its function in a wide range of plant crops.

Moreover, the inventors have further found that the strain of the invention (see example 1): (a) was able to produce siderophores; (b) produced enzymes such as leucine arylamidase, α-glucosidase and α-mannosidase; (c) was a biofilm producer; and (d) is environmental friendly

The production of siderophores as well as of the enzymes leucine arylamidase, α-glucosidase and α-mannosidase activities have beneficious impact on plant growth, development and reproduction.

Siderophores are soluble Fe³⁺ binding agents which can also increase the availability and uptake of iron of the plant. Leucine arylamidase liberates amino acids from polymeric high-molecular-mass compounds providing a source of dissolved organic nitrogen that is an essential nutrient for the plant. α-glucosidase and α-mannosidase act breaking down complex carbohydrates into their monomers, providing a source of sugars to the plant. A biofilm is a community of microbial cells bounded by a polymeric matrix comprising polysaccharides associated with a surface which are reported to be important for soil quality, plant nutrition and plant protection.

Therefore, the strain of the present invention benefits the growth of the bacterized plant.

Altogether the strain of the present invention means a great advance in the field of biopesticides.

Thus, the first aspect of the present invention refers to a strain of Microbacterium esteraromaticum deposited at the “Colección Espanola de Cultivos Tipo” (CECT) under the accession number CECT9167, or a mutant thereof, wherein said mutant is obtained using the CECT9167 of Microbacterium esteraromaticum as starting material and maintains the nematicidal effect of CECT9167. In the present invention the terms “B24” and “CECT9167”, referring to the strain of the invention, are used interchangeably.

The strain of the invention was isolated from an agricultural soil in Almeria (South of Spain) and was deposited, according to the Budapest Treaty, at the CECT in the Universidad de Valencia C. P 46980 Catedratico Agustin Escardino No. 9 Paterna, Valencia (Spain), under the accession number CECT9167. It was deposited by the depositor Futureco Bioscience S. A., Av. Del Cadi 19-23 P. I. Sant Pere Molanta 08799 Olèrdola Barcelona, on the 13 Jul. 2016. The strain was identified by the depositor with the reference B24, and received the accession number CECT9167. It was, in addition, declared viable.

A second aspect of the present invention refers to a bacterial culture comprising the strain as defined in the first aspect of the invention or the mutant thereof.

Due to the pesticidal effect of the strain of the present invention, it can be used as a phytosanitary product. As it is illustrated in the examples below, the strain of the first aspect of the invention can be used as a phytosanitary product according to FAO guidelines, which are the guidelines emitted by Food and Agriculture Organization of the United Nations. The isolated bacteria as defined in the first aspect of the invention may be used as ingredient of a phytosanitary composition.

Therefore, a third aspect of the present invention refers to a composition comprising an effective amount of the strain as defined in the first aspect of the invention, or the bacterial culture as defined in the second aspect of the invention, and one or more agriculturally acceptable compound(s).

A fourth aspect of the invention refers to a method to obtain a mutant of the strain of CECT9167 of Microbacterium esteraromaticum, which comprises using the deposited strain as starting material and applying a genetic engineering technique, for example a DNA recombinant technique, for example mutagenesis, wherein the obtained mutant maintains the activity of the parent deposited strain of controlling a plant nematode and infection in a plant.

A fifth aspect of the invention refers to a process for obtaining a viable cell suspension derived from the strain CECT9167 of M. esteraromaticum, or a mutant thereof, of the first aspect of the invention; the process comprising: (i) inoculating the strain in a suitable culture medium, (ii) subjecting the inoculated culture medium of the step (i) to conditions suitable for growth of the strain, and (iii) optionally subjecting the medium resulting from step (ii) to a concentration step.

As indicated before, the nematicidal effect is due to the fact that the strain of the present invention releases at least certain enzymes and participates in the ISR of the plant. Therefore, a sixth aspect of the invention refers to a supernatant derived from the strain CECT9167 of M. esteraromaticum or a mutant thereof as defined in the first aspect of the invention, said supernatant being obtainable by a process comprising: (i) inoculating the strain in a suitable culture medium; (ii) subjecting the inoculated culture medium to suitable growth conditions; (iii) separating the cells from the culture medium of step (ii); (iv) collecting the supernatant; and (v) optionally subjecting the supernatant to a concentration step.

A seventh aspect of the invention refers to a kit that comprises an effective amount of the strain CECT9167 of Microbacterium esteraromaticum of the first aspect of the invention or the mutant thereof, or the bacterial culture of the second aspect of the invention or the composition of the third aspect of the invention or the supernatant of the sixth aspect of the invention.

An eighth aspect of the present invention refers to the use of the strain CECT9167 of Microbacterium esteraromaticum of the first aspect of the invention or the mutant thereof or the supernatant of the sixth aspect of the invention or the kit of the seventh aspect of the invention for controlling a nematode infection in a plant.

A ninth aspect of the present invention refers to a method for controlling an infection caused by nematodes in a plant comprising applying to a part of a plant or to the substrate used for growing said plant the strain CECT9167 of Microbacterium esteraromaticum of the first aspect of the invention, or the mutant thereof, or the bacterial culture of the second aspect of the invention, or the composition of the third aspect of the invention, or the supernatant of the sixth aspect of the invention, or the kit of the seventh aspect of the invention.

### BRIEF DESCRIPTION OF THE DRAWINGS

FIG. 1 represents egg hatching of M. javanica and M. incognita eggs (50% of each species) after in vitro treatment using M. esteraromaticum strain B24 (at 8.0×10⁸ CFU/ml) and Nemacur® (“chemical”) or a negative control.

FIG. 2 represents egg hatching of M. javanica and M. incognita eggs (50% of each species) after in vitro treatment with M. esteraromaticum strain B24 (at 3×10⁷ CFU/mL), Nemacur® (“chemical”) or a negative control.

FIG. 3 shows egg hatching of M. javanica and M. incognita eggs (50% of each species) after in vitro treatment with M. esteraromaticum B24 (at 5.20×10⁷ CFU/mL), B2538 (6.2×10⁷ CFU/mL) or DSMZ (DSMZ 8609) (at 2.03×10⁷ CFU/mL) strains or a negative control.

FIG. 4 shows survival rate of M. javanica and M. incognita juveniles (J₂) (50% of each species) after in vitro treatment with M. esteraromaticum B24 strain at two concentrations (triangle, 1.4×10⁷ CFUs/mL; square, 2.1×10⁶ CFUs/mL).

FIG. 5 shows survival rate of M. javanica and M. incognita juveniles (J₂) (50% of each species) after in vitro treatment with M. esteraromaticum B24 (at 5.2×10⁷ CFU/mL), B2538 (at 6.2×10⁷ CFU/mL) or DSMZ (DSMZ 8609) (at 2.03×10⁷ CFU/mL) strains.

FIG. 6 represents a comparison of the percentage of efficacy in vitro on egg hatching inhibition of G. rostochiensis and G. paffida eggs of the M. esteraromaticum strain B24 (at 7.0×10⁷ CFU/mL) with respect to the control and a chemical product (Vydate®).

FIG. 7 represents a comparison of the percentage of efficacy in vitro on egg hatch from cysts of G. rostochiensis and G. paffida of the M. esteraromaticum strain B24 applied as TGAI (at 7.0×10⁷ CFU/mL) with respect to the control and a chemical product (Vydate®).

FIG. 8 represents a comparison of the percentage of efficacy in vitro on egg hatch from cysts of G. rostochiensis and G. paffida of the M. esteraromaticum strain B24 applied as OD Formulation at 1% (2.2×10⁸ CFU/mL) with respect to the control and a chemical product (Vydate®).

FIG. 9 represents an HPLC chromatogram at 254 nm of CS of B24-Flask (dashed line), CS of DSM 8609-Flask (dotted line) and compared with culture medium without microbial inoculation (solid line). Relevant different peaks were labelled with arrows.

In all the figures: data represented with different letter (“a”, “b” or “ab”) indicate that there was a statistically significant difference among them (data were subjected to analysis of variance (ANOVA) using R program; treatment means were compared using Fisher's protected least significant difference test (LSD) at P=0.05). “Control”: negative control. “Egg hatch (%)”: percentage of egg hatching.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition. The definitions given herein are included for the purpose of understanding and expected to be applied throughout description, claims and drawings.

The first aspect of the invention refers to an isolated strain of Microbacterium esteraromaticum deposited at the “Colección Espanola de Cultivos Tipo” (CECT) under the accession number CECT9167, or a mutant thereof, wherein said mutant strain is obtained using the CECT9167 of Microbacterium esteraromaticum and maintains the nematicidal effect of CECT9167.

In a particular embodiment of the first aspect of the invention, optionally in combination with any of the embodiments provided below, the isolated strain of Microbacterium esteraromaticum is the M. esteraromaticum strain B24 deposited at the “Colección Espanola de Cultivos Tipo (CECT)” under the accession number CECT9167.

In another embodiment of the first aspect of the invention, optionally in combination with any of the embodiments provided above or below, the strain is a mutant of the strain CECT9167 which maintains the nematicidal effect of the starting strain.

The term “mutant” of the strain CECT9167 is also understood according to the invention as a “variant” of the strain CECT9167.

By using the deposited strain as starting material, the skilled person in the art can routinely, by genetic engineering techniques such as mutagenesis or bacterial recombination techniques, obtain mutants that maintain the herein described relevant features and advantages of the strain of the invention. In an embodiment of the invention, the mutant is a genetically modified mutant obtained by random mutagenesis (i.e. using chemical or physical agents) or by site-directed mutagenesis, combinatorial mutagenesis or insertional mutagenesis. In another embodiment of the first aspect of the present invention, the mutant is obtained by using recombinant technology, for example using transformation (for example, by electroporation, heat-shock or using divalent cation solutions, such as calcium chloride), transduction or conjugation techniques. By using recombinant technology a plasmid can be included in the bacterial strain, said plasmid can comprise antibiotic resistant genes or genes that serve for the selection of the mutant. Examples of genetic engineering techniques can be found in Sambrook, J. and Russell, D. W. “Molecular Cloning: A Laboratory Manual”, Chapter 13, “Mutagenesis”, Cold Spring Harbor, 3rd Ed, 2001.

As mentioned above, the mutant provided by the present invention has to maintain the effect shown by the strain CECT9167, i.e., the anti-nematode activity. In the present invention the term “maintain”, when referred to the mutant, means that it has to display the anti-nematode activity. Of course, as a consequence of the mutation in the strain, the resulting mutant encompassed by the present invention can display the activity more efficiently than the strain CECT9167 of Microbacterium esteraromaticum.

In order to determine whether the mutant maintains the broad anti-nematode profile, several well-known protocols can be followed. These methods are commonly based on the analysis of the growth capacity of the pathogen in contact with the strain, or the assessment of the severity of the disease caused by the pathogen infection after the exposition to the strain. The protocols that are included in the examples herein for the determination of the activity are illustrative and non-limitative examples. Briefly, they are based on in vitro nematode egg-hatching and nematode survival; and in vivo nematode reproduction analysis.

In a particular embodiment of the first aspect of the invention, optionally in combination with any of the embodiments provided above or below, the nematicidal effect is against at least a nematode of the genus Meloidogyne, Globodera and/or Pratylenchus. In another embodiment, optionally in combination with any of the embodiments provided above or below, the nematicidal effect is against at least a nematode of the genus Meloidogyne and Globodera. In another embodiment, optionally in combination with any of the embodiments provided above or below, the nematicidal effect is against one or more of Meloidogyne incognita, Meloidogyne javanica, Globodera rostochiensis and Globodera paffida. In another embodiment, the strain as defined in the first aspect of the invention has a nematicidal effect against Meloidogyne incognita, Meloidogyne javanica, Globodera rostochiensis and Globodera paffida. In another embodiment, optionally in combination with any of the embodiments provided above or below, the nematicidal effect is against one or more of Pratylenchus penetrans, Pratylenchus fallax, Pratylenchus coffeae, Pratylenchus loosi, and Pratylenchus vulnus.

In another embodiment of the first aspect of the invention, optionally in combination with any of the embodiments provided above or below, the strain as defined in the first aspect of the invention has a nematicidal effect against Meloidogyne incognita, Meloidogyne javanica, Globodera rostochiensis and Globodera paffida.

In another embodiment of the first aspect of the invention, optionally in combination with any of the embodiments provided above or below, the mutant of the strain CECT9167 of Microbacterium esteraromaticum has a genomic sequence identity of at least 99.8% or 99.9% with the strain CECT9167 of Microbacterium esteraromaticum.

In the present invention the term “identity” refers to the percentage of residues that are identical in the two sequences when the sequences are optimally aligned. If, in the optimal alignment, a position in a first sequence is occupied by the same amino acid residue as the corresponding position in the second sequence, the sequences exhibit identity with respect to that position. The level of identity between two sequences (or “percent sequence identity”) is measured as a ratio of the number of identical positions shared by the sequences with respect to the size of the sequences (i.e., percent sequence identity=(number of identical positions/total number of positions)×100).

A number of mathematical algorithms for rapidly obtaining the optimal alignment and calculating identity between two or more sequences are known and incorporated into a number of available software programs. Examples of such programs include the MATCH-BOX, MULTAIN, GCG, FASTA, and ROBUST programs for amino acid sequence analysis, among others. Preferred software analysis programs include the ALIGN, CLUSTAL W, and BLAST programs (e.g., BLAST 2.1, BL2SEQ, and later versions thereof).

For amino acid sequence analysis, a weight matrix, such as the BLOSUM matrixes (e.g., the BLOSUM45, BLOSUM50, BLOSUM62, and BLOSUM80 matrixes), Gonnet matrixes, or PAM matrixes (e.g., the PAM30, PAM70, PAM120, PAM160, PAM250, and PAM350 matrixes), can be used in determining identity.

The BLAST programs provide analysis of at least two sequences, either by aligning a selected sequence against multiple sequences in a database (e.g., GenSeq), or, with BL2SEQ, between two selected sequences. BLAST programs are preferably modified by low complexity filtering programs such as the DUST or SEG programs, which are preferably integrated into the BLAST program operations. If gap existence costs (or gap scores) are used, the gap existence cost preferably is set between about −5 and −15. Similar gap parameters can be used with other programs as appropriate. The BLAST programs and principles underlying them are further described in, e.g., Altschul et al., “Basic local alignment search tool”, 1990, J. Mol. Biol, v. 215, pages 403-410.

For multiple sequence analysis, the CLUSTAL W program can be used. The CLUSTAL W program desirably is run using “dynamic” (versus “fast”) settings. Sequences are evaluated using a variable set of BLOSUM matrixes depending on the level of identity between the sequences. The CLUSTAL W program and underlying principles of operation are further described in, e.g., Higgins et al., “CLUSTAL V: improved software for multiple sequence alignment”, 1992, CABIOS, 8(2), pages 189-191.

In another embodiment of the first aspect of the invention, optionally in combination with any of the embodiments provided above or below, the mutant of the strain CECT9167 of Microbacterium esteraromaticum has an average nucleotide identity (ANI) of at least 99.8% or 99.9% with the strain CECT9167 of Microbacterium esteraromaticum.

In another embodiment of the first aspect of the invention, optionally in combination with any of the embodiments provided above or below, the method to determine the ANI of the mutant of the invention is the method “ANIm” described in Richter M, et al. 2015 JSpeciesWS: a web server for prokaryotic species circumscription based on pairwise genome comparison. Bioinformatics. 2015 Nov. 16. pii: btv681.

In another embodiment of the first aspect of the invention, optionally in combination with any of the embodiments provided above or below, the method to obtain a mutant of the strain of the invention, for example with a genomic sequence identity of at least 99.8% or 99.9% with the strain CECT9167 of Microbacterium esteraromaticum are the mutagenic methods described in Aubert et al., “A Markerless Deletion Method for Genetic Manipulation of Burkholderia cenocepacia and Other Multidrug-Resistant Gram-Negative Bacteria” Methods Mol Biol 2014; 1197:311-27.

A second aspect of the invention refers to a bacterial culture comprising the strain as defined in the first aspect of the invention.

In an embodiment of the second aspect of the invention, optionally in combination with any of the embodiments provided above or below, the bacterial culture comprises a viable strain as defined in the first aspect of the invention.

In an embodiment of the second aspect of the invention, optionally in combination with any of the embodiments provided above or below, the bacterial culture is an inoculation product.

By “inoculation product” it is understood a product obtained after inoculating the strain in a suitable culture medium, subjecting the inoculated culture medium to suitable growth conditions.

In another embodiment of the second aspect of the invention, optionally in combination with any of the embodiments provided above or below, the bacterial culture comprises the strain of the invention inactivated.

The term “inactivated” means that the micro-organism is not able to form colonies. In one embodiment, the inactivated micro-organisms have the cell membrane intact or broken.

In another embodiment of the second aspect of the invention, optionally in combination with any of the embodiments provided above or below, the inoculation product comprises the strain of the invention inactivated.

By “inoculation product comprising the strain of the invention inactivated” refers to a product obtained after inoculating the strain in a suitable culture medium, subjecting the inoculated culture medium to suitable growth conditions, and then inactivating the strain.

With a view to practical use in pest control, pesticide agents are usually formulated into compositions also including agriculturally acceptable compounds. Therefore, a third aspect of the present invention refers to a composition comprising an effective amount of the strain as defined in the first aspect of the invention, or the bacterial culture as defined in the second aspect of the invention, and one or more agriculturally acceptable compounds.

The term “effective amount” as used herein, means an amount of the strain M. esteraromaticum CECT9167 as defined in the first aspect of the invention, high enough to provide the desired benefit, either the treatment or prevention of the plant disease, but low enough to avoid serious side-effects. The particular dose of compound administered according to this invention will of course be determined by the particular circumstances surrounding the case, including the compound administered, the route of administration, the particular condition being treated, and the similar considerations.

In an embodiment of the third aspect of the invention, optionally in combination with any of the embodiments provided above or below, the strain is present at a concentration from 1.0×10⁵ CFUs/mL to 1.0×10¹² CFUs/mL. In another embodiment of the third aspect of the invention, the strain is present at a concentration of 1.0×10⁵ CFUs/mL, 1.0×10⁶ CFUs/mL, 1.0×10⁷ CFUs/mL, 1.0×10⁸ CFUs/mL, 1.0×10⁹ CFUs/mL, 1.0×10¹⁰ CFUs/mL, 1.0×10¹¹ CFUs/mL or 1.0×10¹² CFUs/mL.

For the purposes of the invention, any ranges given include both the lower and the upper end-points of the range.

The amount indicated as CFU/mL relates to the colony forming units (CFU or CFUs) of the strain of the invention per milliliter or gram of the composition.

In another embodiment of the third aspect of the invention, optionally in combination with any of the embodiments provided above or below, the composition is a phytosanitary composition.

In another embodiment of the third aspect of the invention, optionally in combination with any of the embodiments provided above or below, the composition comprises at least one additional pesticide.

In another embodiment of the third aspect of the invention, optionally in combination with any of the embodiments provided above or below, the additional pesticide is selected from the group consisting of another bacterial strain with pesticide properties, a fungicide, a bactericide, an herbicide, an insecticide or a chemical nematicide. Said additional pesticide does not have to adversely affect the activity/viability of the strain of the invention included in the composition.

In the present invention, the term “pesticide” is understood by its usual meaning in the field of agronomy as a product intended to kill, repel, regulate or disrupt the growth of living organisms that are considered pests. Clearly, due to the nature of the strain CECT9167 of M. esteraromaticum or of the mutant thereof, herein it is understood that “pesticide” is a biological or ecological (organic) pesticide, also called biopesticide. In the scope of the present invention, the term “pesticide” would have the same meaning as the term “phytosanitary”.

In an embodiment of the third aspect of the invention, optionally in combination with any of the embodiments provided above or below, the composition can be in the form of a solution, a pellet, a suspension, a lyophilized compositions or other dried compositions (for example freeze dried composition). The lyophilized or dried composition can be reconstituted with a liquid carrier prior to its use or directly used. The composition may be prepared according to various formulations suitable for phytosanitary uses, for example, chosen from the group consisting of formulations of the following type: liquid intended for use without dilution (AL), powder intended for use without dilution (AP), encapsulated granule (CG), contact liquid or gel (CL), contact powder (CP), powdering powder (DP), emulsifiable concentrate (EC), emulsifiable granule (EG), oil type emulsion (EO), water type emulsion (EW), fine granule (FG), macrogranules (GG), emulsifiable gel (GL), powder for spraying (GP), granules (GR), grease (GS), water-soluble gel (GW), microemulsion (ME), microgranules (MG), water-dilutable concentrated suspension (OF), oil dispersion (OD), water-miscible suspension (OL), powder for dispersion in oil (OP), concentrated in gel or paste form (PC), sticks (for agri-pharmaceutical use) (PR), concentrated suspension (SC), suspoemulsion (SE), water-soluble granules (DG), soluble concentrate (SL), film-forming oil (SO), water-soluble powder (SP), water-soluble tablets (ST), tablets (TB), water-dispersible granules (WG), wettable powder (WP), water-dispersible tablets (WT) (the code consisting of two capital letters corresponding to the international codes for phytosanitary formulations).

In an embodiment of the third aspect of the invention, optionally in combination with any of the embodiments provided above or below, the composition is an OD composition.

In an embodiment of the third aspect of the invention, optionally in combination with any of the embodiments provided above or below, the composition is an OD formulation and comprises a concentration of the strain as defined in the first aspect of the invention in the range from 1.0×10⁵ to 1.0×10¹² CFU/mL (1.0×10⁵, 1.0×10⁶, 1.0×10⁷, 1.0×10⁸, 1.0×10⁹, 1.0×10¹⁰, 1.0×10¹¹ or 1.0×10¹² CFU/mL).

In an embodiment of the third aspect of the invention, optionally in combination with any of the embodiments provided above or below, the composition is an OD formulation and comprises a concentration of the strain as defined in the first aspect of the invention of 10¹⁰ CFU/mL.

In an embodiment of the third aspect of the invention, optionally in combination with any of the embodiments provided above or below, the composition is an OD formulation and comprises a concentration of the strain as defined in the first aspect of the invention in the range from 1.0×10⁵ to 1.0×10¹² CFU/mL and also comprises a vegetable oil, an organic ester, silica, and a high molecular weight copolymer.

In an embodiment of the third aspect of the invention, optionally in combination with any of the embodiments provided above or below, the composition comprises an oily ingredient. In another embodiment, the oily ingredient is a vegetable oil. In another embodiment, the vegetable oil is soy oil.

In an embodiment of the third aspect of the invention, optionally in combination with any of the embodiments provided above or below, the composition comprises soy oil, C18 ethoxilated fatty acid, silica and polyacrylate crosspolymer.

In another embodiment of the third aspect of the invention, optionally in combination with any of the embodiments provided above or below, the composition is an OD composition which comprises soy oil, C18 ethoxilated fatty acid, silica, polyacrylate crosspolymer and comprises a concentration of the strain as defined in the first aspect of the invention in the range from 1.0×10⁵ to 1.0×10¹² CFU/mL.

In another embodiment of the third aspect of the invention, optionally in combination with any of the embodiments provided above or below, the composition is an OD composition which consists of 70% of soy oil, 20% of C18 ethoxilated fatty acid, 1% silica, polyacrylate crosspolymer and 8% of the bacterial strain of the present invention; in another embodiment, the final concentration of the bacteria is 1×10¹⁰ CFU/mL.

“Agriculturally acceptable compounds” refers to those compounds and/or materials, which are suitable for use in agriculture. In general, said compounds should be non-toxic to humans and preferably should be environment-friendly.

In a particular embodiment of the third aspect of the invention, optionally in combination with any of the embodiments provided above or below, the compositions of the invention may contain compounds for improving the adhesion of the strains in the plants to be treated, as well as phytostrengthener compounds, nutrients, wetting agents, stabilizers, osmotic protectors, antioxidants, sunscreens, buffering compounds or combinations thereof.

Examples of adhesion products are gelatin, starch, pectins, alginates and various types of gums such as xanthan. Many of these compounds are also wetting agents. In the case of sunscreens, Congo red, calcium carbonate and wax emulsions can be used. The phytostrengtheners are compounds that can facilitate make crops develop robustness or tolerance towards pathogens or adverse environmental conditions, for example, jasmonic acid analogues and some plant defense stimulants such as harpins, chitosans, and laminarins. Additionally, examples of osmotic protectors are trehalose, betaines and amino acids. Finally, ascorbic acid and glutathione are included among antioxidants.

The compositions of the third aspect of the invention can be prepared by routine protocols, such as by mixing the different ingredients.

A fourth aspect of the invention refers to a method to obtain a mutant of the strain of CECT9167 of Microbacterium esteraromaticum, which maintains the nematicidal effect of CECT9167 comprising the step of subjecting the strain of CECT9167 to a genetic engineering technique.

In an embodiment of the fourth aspect of the invention, optionally in combination with any of the embodiments provided above or below, the genetic engineering technique is mutagenesis, such as random mutagenesis (i.e. using chemical or physical agents), by site-directed mutagenesis, combinatorial mutagenesis or insertional mutagenesis; or a recombinant technique, such as transformation (for example, by electroporation, heat-shock or using divalent cation solutions, such as calcium chloride), transduction or conjugation techniques. In another embodiment of the fourth aspect of the invention, the genetic engineering technique is mutagenesis.

In an embodiment of the fourth aspect of the invention, optionally in combination with any of the embodiments provided above or below, the mutant maintains the production of siderophores, enzymes, such as leucine arylamidase, alpha-gluscosidase and/or mannosidase; and/or the production of biofilm.

In an embodiment of the fourth aspect of the invention, optionally in combination with any of the embodiments provided above or below, the mutant of the strain CECT9167 of Microbacterium esteraromaticum has a genomic sequence identity of at least 99.8% or 99.9% with the strain CECT9167 of Microbacterium esteraromaticum.

A fifth aspect of the invention refers to a process for obtaining a viable cell suspension derived from the strain CECT9167 of M. esteraromaticum or a mutant thereof of the first aspect of the invention, the process comprising: (i) inoculating the strain in a culture medium, (ii) subjecting the inoculated culture medium of the step (i) to conditions suitable for growth of the strain, and (iii) optionally subjecting the medium resulting from step (ii) to a concentration step.

The term “derived from the strain CECT9167” means that the suspension is obtained from the strain as defined in the first aspect of the present invention.

The strain of the invention may be inoculated in the culture medium at a final concentration comprised from 3 to 7% v/v, for example from 5 to 7% v/v, for example of 5% v/v. Preferably the inoculated culture is in an exponential growth phase. Suitable culture media for the growth of the strain of the invention are synthetic media, such as LB (lysogenic broth) and PM (saline production medium), or media of plant origin such as molasses (e.g. from sugar cane, beets and others). Suitable conditions for strain's growth are temperatures comprised from 6 to 42° C., pH comprised 4 to 10 (for example 7 to 10), and oxygen concentration comprised from 10 to 100%. In a particular embodiment, the conditions for the growth of the strain are temperatures of 28-30° C., pH of 7-8, and oxygen concentration of 50%. During the growth of the strain of the invention stirring can be used.

In another embodiment of the fifth aspect of the invention, optionally in combination with any of the embodiments provided above or below, cells are separated from the medium to obtain a concentrated suspension. Suitable separation techniques include centrifugation or filtration of the culture. Carrying out the centrifugation of the culture, for example, at a minimum of 5000 rpm, cells are obtained in the pellet, which are resuspended in part of the culture medium or in a suitable buffered medium such that the strain concentration is approximately about 1×10⁵-1×10¹² CFU/mL (10⁵ CFU/mL, 10⁶ CFU/mL, 10⁷ CFU/mL, 10⁸ CFU/mL, 10⁹ CFU/mL, 10¹⁰ CFU/mL, 10¹¹ CFU/mL or 10¹² CFU/ml).

Once the suspension is obtained, it may be subjected to a dehydration step. Dehydration can be carried out through a lyophilisation process. Alternatively, the suspension can be dehydrated by fluidised bed drying. Another option is to dehydrate the suspension by spray drying or drying in an oven under vacuum. In this regard, another advantageous feature of the strain of the invention is that it exhibits high resistance to dehydrating processes, which are routine in obtaining microorganisms on an industrial scale. In order to improve cell viability, an inert osmotic protector ingredient can be added to the suspension before carrying out the dehydration process.

In another particular embodiment of the fifth aspect of the invention, optionally in combination with any of the embodiments provided above or below, the process comprises re-suspending the cells resulting from the separation step in a suitable buffer to yield a cell concentrated suspension.

The cells obtained with the fifth aspect of the invention may then be used directly, resuspended to a desired density, subjected to dehydration or disrupted to obtain a cell free extract.

A sixth aspect of the present invention refers to a supernatant derived from the strain CECT9167 of M. esteraromaticum or a mutant thereof as defined in the first aspect of the invention, said supernatant being obtainable by a process comprising: (i) inoculating the strain in a suitable culture medium; (ii) subjecting the inoculated culture medium to suitable growth conditions; (iii) separating the cells from the culture medium of step (ii); (iv) collecting the supernatant; and (v) optionally subjecting the supernatant to a concentration step.

In a particular embodiment of the sixth aspect of the invention, optionally in combination with any of the embodiments provided above or below, the supernatant is from the strain CECT9167 of M. esteraromaticum or a mutant thereof as defined in the first aspect of the invention.

In a particular embodiment of the sixth aspect of the invention, optionally in combination with any of the embodiments provided above or below, the supernatant is produced by the strain CECT9167 of M. esteraromaticum or a mutant thereof as defined in the first aspect of the invention, and comprises N-acetyl-β-glucosaminidase, leucine arylamidase, α-glucosidase and α-mannosidase, for example, when the culture medium is liquid medium, for example is tryptic soy broth (TSB).

Another aspect of the present invention provides a cell extract derived from the strain CECT9167 of M. esteraromaticum or a mutant thereof as defined in the first aspect of the invention, said cell extract being obtainable by a process comprising: (i) inoculating the strain in a suitable culture medium; (ii) subjecting the inoculated culture medium to suitable growth conditions; (iii) separating the cells from the culture medium of step (ii); (iv) collecting the cells and (v) disrupting the cells, (vi) separating the cell extract from cell debris, (vii) collecting the cell extract, and (viii) optionally subjecting the cell extract to a concentration process.

Suitable disrupting means are known by the skilled person and may include physical disruption, for example freeze-thaw or French Press, or chemical disruption, for example by addition of lysozyme. Suitable separation means have been described above. The cell free extract may also be obtained from a cell suspension as defined above, preferably containing metabolite-containing supernatant.

The supernatant of the sixth aspect of the invention or the cell extract obtained by the methods described above, could be used and/or included in an appropriated composition directly or subjected to a concentration step to reach a more suitable composition. Thus, in an embodiment of said aspects of the invention the step of concentration of the supernatant or, alternatively, of the cell extract can be performed by dehydration, filtration, ultra-filtration, centrifugation, ultra-centrifugation, precipitation or chromatography.

A seventh aspect of the present invention relates to a kit that comprises an effective amount of the strain, or the mutant thereof as defined in the first aspect of the invention, or the bacterial culture of the second aspect of the invention or the composition of the third aspect of the invention or the supernatant of the sixth aspect of the invention.

All the embodiments provided above for the strain of the first aspect of the invention, the bacterial culture of the second aspect of the invention and the composition of the third aspect of the invention are also embodiments of the kit of the seventh aspect of the invention.

In an embodiment of the seventh aspect of the invention, optionally in combination with any of the embodiments provided above or below, the kit may comprise a weekly, monthly, or other periodic dose of the strain of the first aspect of the invention. As an illustrative example, a kit comprising a weekly dose may comprise seven discrete compositions comprising the strain (seven daily doses). As another example, a kit comprising a monthly dose may comprise thirty compositions comprising the strain of the first aspect of the invention.

In case the strain of the first aspect of the invention is lyophilized, the kit of the eighth aspect of the invention can contain a resuspension agent, such as water.

In another embodiment of the seventh aspect of the invention, optionally in combination with any of the embodiments provided above or below, the kit comprises means to facilitate dosing compliance. For example, the kits may be particularly advantageous for the purpose of ensuring that the person who used it is performing the correct administration of the effective amount of the strain of the invention to the plant on the appropriately prescribed schedule. Blister cards or other containing devices appropriately configured may be particularly suitable for clearly illustrating sequence or timing of administration of the various components. The kit may be obtained as one card, or cases of four, six, seven (e.g., a weekly supply), or eight cards co-packaged together. Additionally, monthly or other types of kits may be obtained.

The kit that comprises the isolated strain, or the mutant, of the first aspect of the invention can comprise any means that allows the correct culture of the strain of the invention, such as culture medium, supplements or antibiotics, as well as instructions for the correct preparation and/or application to the plant.

An eighth aspect of the invention refers to the use of the strain CECT9167 of Microbacterium esteraromaticum or the mutant thereof of the first aspect of the invention, or the supernatant of the sixth aspect of the invention, or the kit of the seventh aspect of the invention for controlling a nematode infection in a plant.

A ninth aspect of the present invention refers to a method for controlling an infection caused by nematodes in a plant comprising applying to a part of a plant or to the substrate used for growing said plant the strain CECT9167 of Microbacterium esteraromaticum of the first aspect of the invention or the mutant thereof, or the supernatant of the sixth aspect of the invention, or the kit of the seventh aspect of the invention.

The strain of the eighth and ninth aspects of the invention can be used as a bacterial culture or as a composition.

All the embodiments provided above for the strain of the first aspect of the invention, the bacterial culture of the second aspect of the invention and the composition of the third aspect of the invention are also embodiments of the eighth and ninth aspects of the invention.

In an embodiment of the ninth aspect of the invention, optionally in combination with any of the embodiments provided above or below, the strain CECT9167 of M. esteraromaticum, or a mutant thereof as defined in the first aspect of the invention is administered to the plant at a dosage regime of 10⁶-10⁸ CFU/g or 10⁶-10⁸ CFU/mL per day once during several weeks, for example 10⁶-10⁸ CFU/g or 10⁶-10⁸ CFU/mL 1-6 times in several weeks, or for example 10⁶-10⁸ CFU/g or CFU/mL 1-4 times per treatment.

The dose may be adapted according to the composition of the third aspect of the invention and the formulation of the composition which is used and also according to the weather conditions, any resistance phenomena or other natural factors, the nature of the treatment or the degree of infestation, and according to the plants or sites to be treated.

In an embodiment of the ninth aspect of the invention, optionally in combination with any of the embodiments provided above or below, the strain, the bacterial culture or the composition is applied to plant nursery trays, during seed treating, seed dressing, seed disinfection, seedling root dipping treatment, planting pit treatment, plant foot treatment, planting row treatment, surface spraying, soil incorporation, drip irrigation, or by application to a water culture medium in hydroponic culture.

The term “part of a plant” includes any segment of the plant, such as the root, stem, leaves and seeds.

In an embodiment of the ninth aspect of the invention, optionally in combination with any of the embodiments provided above or below, the part of the plant treated is the root system (root).

In an embodiment of the ninth aspect of the invention, optionally in combination with any of the embodiments provided above or below, the part of the plant treated is the seed. This treatment can be achieved by ordinary methods; for example, a method wherein seeds are dipped in the composition of the third aspect of the invention, coating the seeds.

The term “seed” includes what is called seeds, and also plant bodies for vegetative propagation such as bulbs, tubers and seed potato.

The treatment of the plant, i. e. root and/or the seeds, allows the nematicidal action of the strain of the invention against root-nematodes.

The term “plant” comprises all plant species cultivated by humans, in particular those intended for food or for animal feed, such as cereals, fodder, vegetable, fruit crops, vines, and/or for the supply of wood for all purposes (such as heating, housing construction furniture, and/or ornamentation). Example of plants include cereals (for example, rice, barley, wheat, rye, oat and corn), beans (for example, soybean, azuki bean, broad bean, peas and peanuts), fruit trees/fruits (for example, apples, pears, citruses, grapes, peaches, apricots, cherries, olive, nuts, almonds, bananas, berries and strawberries), vegetables (for example, tomato, cabbage, spinach, broccoli, lettuce, onion, garlic, leek and pepper), root crops (for example, potato, carrot, sweet potato, radish, lotus root and turnip), industrial crops (for example, cotton, hemp, paper mulberry, mitsumata, rape, beet, hop, sugarcane, sugar beet, rubber, coffee, tobacco, tea), pepos (for example, pumpkin, cucumber, watermelon, melon), pasture plants (for example, orchardgrass, sorghum, Thimothy-grass, clover, alfalfa), lawn grasses (for example, mascarene grass, bentgrass), crops for flavorings (for example, lavender, rosemary, thyme, parsley, basilica, mint, coriander, pepper, ginger), and flower plants (for example, Chrysanthemum, rose, orchids).

In an embodiment of the eighth or the ninth aspect of the invention, optionally in combination with any of the embodiments provided above or below, the plant is tomato, cucumber or potato plant.

The term “substrate” comprises any support for cultivating a plant, and the material therefor is not particularly limited, as far as the plant can grow therein; for example, nursery mats, water, sand, soil, vermiculite, cotton, paper, diatomaceous earth, agar, gel substances, polymeric substances, rock wool, glass wool, wood chips, barks and pumice.

In an embodiment of the ninth aspect of the invention, optionally in combination with any of the embodiments provided above or below, the method of application is performed in the vicinity of the plant or a nursery bed for raising seedlings.

In an embodiment of the ninth aspect of the invention, optionally in combination with any of the embodiments provided above or below, the method of application is performed directly in the plant. In an embodiment, the method of application is performed on the root system and/or the seeds.

The examples below demonstrate the use of a strain of M. esteraromaticum as a bio-pesticide, in the treatment of nematode infection.

In an embodiment of the eighth and ninth aspects of the invention, optionally in combination with any of the embodiments provided above or below, the use of the eighth aspect and the method of the ninth aspect are for controlling the colonization of pest nematodes or treating infested plants. In particular, the invention relates to uses for controlling those plant pests. The term “treating” therefore is referred to infested plants. The term “control” comprises preventing infestations of the plants by said pests, repelling or eliminating said pests.

In an embodiment of the eighth and ninth aspects of the invention, optionally in combination with any of the embodiments provided above or below, the use of the eighth aspect and the method of the ninth aspect are for treating a plant infected by pest nematodes.

In an embodiment of the eighth and ninth aspects of the invention, optionally in combination with any of the embodiments provided above or below, the nematode is a root-knot nematode or a cyst nematode.

In an embodiment of the eighth and ninth aspects of the invention, optionally in combination with any of the embodiments provided above or below, the nematode is of the genus Meloidogyne, such as Meloidogyne incognita (southern root-knot nematode), Meloidogyne javanica (Javanese root-knot nematode), Meloidogyne hapla (northern root-knot nematode), and Meloidogyne arenaria (peanut root-knot nematode); nematodes of the genus Globodera, such as Globodera rostochiensis (golden nematode) and Globodera pallida (potato cyst nematode); nematodes of the genus Ditylelenchus, such as Ditylelenchus destructor (potato rot nematode) and Ditylelenchus dipsaci (bulb and stem nematode); nematodes of the genus Pratylenchus, such as Pratylenchus penetrans (cobb root-lesion nematode), Pratylenchus fallax (chrysanthemum root-lesion nematode), Pratylenchus coffeae (coffee root-lesion nematode), Pratylenchus loosi (tea root-lesion nematode), and Pratylenchus vulnus (walnut root-lesion nematode); nematodes of the genus Heterodera, such as Heterodera glycines (soybean cyst nematode) and Heterodera shachtoii) (sugar beet cyst nematode); nematodes of the genus Aphelenchoides, such as Aphelenchoides besseyi (rice white-tip nematode), Aphelenchoides ritzemabosi (chrysanthemum foliar nematode), and Aphelenchoides fragarieae (strawberry nematode); nematodes of the genus Aphelenchus, such as Aphelenchus avenae (mycophagous nematode); nematodes of the genus Radopholus, such as Radopholus similis (burrowing nematode); nematodes of the genus Tylenchulus, such as Tylenchulus semipenetrans (citrus nematode); nematodes of the genus Rotylenchulus, such as Rotylenchulus reniformis (reniform nematode); or nematodes that occur in trees, such as Bursaphelenchus xylophilus (pine wood nematode).

In an embodiment of the eighth and ninth aspects of the invention, optionally in combination with any of the embodiments provided above or below, the nematode is selected of the genus Meloidogyne; or alternatively the nematode is of the genus Globodera.

In an embodiment of the eighth and ninth aspects of the invention, optionally in combination with any of the embodiments provided above or below, the nematode is selected form the group consisting of Meloidogyne incognita, Meloidogyne javanica, Globodera rostochiensis and Globodera pallida; or, alternatively, the nematode is Meloidogyne incognita; or, alternatively, the nematode is Meloidogyne javanica; or, alternatively, the nematode is Globodera rostochiensis; or, alternatively, the nematode is Globodera pallida.

In the present invention the term “infection” comprises asymptomatic infection or symptomatic infection of the plant caused by a nematode.

In an embodiment of the eighth and ninth aspects of the invention, optionally in combination with any of the embodiments provided above or below, the control can be performed in any plant.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

### Example 1: Characterization of the M. esteraromaticum Strain B24

1.1 Species Identification:

Materials and Methods

The strain B24 of M. esteraromaticum was isolated in an agricultural soil under organic farming regime from Almeria (South of Spain). Identification of the bacterial isolate was achieved using 16S rRNA gene sequence analysis.

The 16S rRNA gene sequencing was performed as described next. A PCR reaction mix, which consisted of 10 μL Phire Green Hot Start II Master Mix (Thermo Fisher Scientific), 0.8 μL primer 8f 10 μM (SEQ ID NO:1), 0.8 μL primer 1492r 10 μM (SEQ ID NO:2), 2 μL template (a single colony picked from a nutritive agar plate, resuspended in 50 μL sterile distilled water, boiled for 10 min at 98° C., and chilled to 4° C.) and 6.4 μL nanopure water, was amplified according to the following program (direct PCR): 98° C. 5 min; 35 cycles of 98° C. 5 s, 58.5° C. 5 s and 72° C. 20 s, and 72° C. 20 s. The PCR product was purified using the EZNA Cycle Pure Kit (Omega Bio-Tek) according to manufacturer's instructions. The purified PCR product was sequenced by an external sequencing service (Secugen) using the primer 1492r (SEQ ID NO: 2) and the Sanger method with BigDye® Terminator v3.1 (Applied Biosystems) according to manufacturer's instructions. The sequencing revealed a sequence (SEQ ID NO: 3) which was 100% identical to that of several M. esteraromaticum strains (NCBI accession numbers MG705679.1, JF496416.1, JF496262.1, AB355700.1, AB099658.1, AB099656.1; database accession date Oct. 1, 2018).

1.2 Characterization of the Enzymatic Activities

Materials and Methods

The enzymatic activities were determined using API® ZYM (bioMérieux, Madrid, Spain), following the manufacturer's instructions. The activities of the following enzymes were tested (in braquets their corresponding substrate): Alkaline phosphatase (2-naphthyl phosphate), Esterase C 4 (2-naphthyl butyrate), Esterase Lipase C 8 (2-naphthyl caprylate), Lipase C 14 (2-naphthyl myristate), Leucine arylamidase (L-leucyl-2-naphthylamide), Valine arylamidase (L-valyl-2-naphtylamide), Cystine arylamidase (L-cystyl-2-naphtylamide), Trypsin (N-benzoyl-DL-arginine-2-naphtylamide), α-chymotrypsin (N-glutaryl-phenylalanine-2-naphtylamide), Acid phosphatase (2-naphtyl phosphate), Naphtol-AS-BI-phosphohydrolase (Naphtol-AS-BI-phosphate), α-galactosidase (6-Br-2-naphthyl-αD-glucopyranoside), β galactosidase (2-naphtyl-βD-glucopyranoside), β-glucuronidase (Naphthol-AS-BI-βD-glucuronide), α-glucosidase (2-naphtyl-αD-glucopyranoside), β-glucosidase (6-Br-2-naphthyl-βD-glucopyranoside), N-acetyl-β-glucosaminidase (1-naphthyl-N-acetyl-βD-glucosaminide), α-mannosidase (6-Br-2-naphtyl-αD-mannopyranosido) and α-fucosidase (2-naphtyl-αL-fucopyranoside).

Results:

B24 produced leucine arylamidase, α-glucosidase and α-mannosidase. There were 13 negatives enzymatic reactions, therefore, B24 did not produce the others tested enzymes in the assay conditions.

1.3: Siderophore Production

Materials and Methods

Production of ferric ion chelates was detected using Chrome Azurol S dye (CAS) methodology (Schwyn B and Neilands J B “Universal chemical assay for the detection and determination of siderophores” 1987 Anal Biochem 160(1): 47-56) with some modifications. CAS solution was made by dissolving 60.5 mg CAS powder (Sigma-Aldrich) in 50 ml distilled water and 10 ml of Fe(III) solution (1 mM FeCl₃.6H₂O, 10 Mm HCl) were added. Under stirring this solution was slowly mixed with 72.9 mg hexadecyltrimethylammonium bromide (HDTMA, Sigma-Aldrich), dissolved in 40 ml water and the resultant dark blue solution was autoclaved to sterilize and stored in the dark.

A CAS assay in liquid media was performed performing the following method: 900 μl of overnight bacterial cultures (grown in Luria-Bertani (LB) medium at 28° C. and 200 rpm) were mixed with 100 μl of the CAS assay solution in a glass tube. Mixtures were allowed to incubate for 30 min at room temperature and compared to an uninoculated media control.

A CAS agar assay was performed performing the following method: overnight bacterial cultures were grown at 28° C. for 24h on King B plates (20 g/L casein peptone, 1.5 g/L K₂HPO₄, 1.5 g/L MgSO₄.7H₂O, 10 ml glycerol, and 15 g/L agar) containing 1/10 of the CAS solution.

Results:

Following CAS assay in agar and liquid media, it was determined that B24 was able to produce siderophores. Both assays showed the change of dark blue to orange color after incubation with the bacteria strain B24 (in the CAS agar assay an orange halo formed around microbial colonies was indicative of siderophores excretion), while no change was observed after incubation with other bacteria that did not produced siderophores.

1.4: Biofilm Formation

Materials and Methods

The strains used in this study consisted of two M. esteraromaticum isolates from the Futureco Bioscience collection: the strain of the invention (B24) and the strain B2538; and a reference strain, the M. esteraromaticum strain DSM 8609 that was purchased form the Deutsche Sammlung von Mikroorganismen and Zellkulturen GmbH (DSMZ) collection.

To determine biofilm formation, overnight cultures of each isolate were grown in LB at 28° C. on a rotary shaker. Next day, cultures were diluted into fresh LB to give an OD₆₀₀ of 0.1. Sterile untreated 96-well microtiter plates were inoculated with 200 μL of the bacterial suspensions and incubated for 48 h at 28° C. Prior to biofilm quantification, cell growth in each well was estimated by measuring the optical density at 620 nm (OD₆₂₀) using a plate reader (Multilabel Plater Reader HALO LED96, Dynamica). Quantification of the biofilm biomass was performed by crystal violet (CV) staining. Briefly, wells containing adhered cells were washed three times with water, fixed at 60° C. for 1 hour and stained during 15 minutes with 200 μL of 0.1% CV. The stained biofilms were rinsed with distilled water, allowed to dry for 30 min at 37° C. and then extracted with 200 μL of 95% ethanol. The amount of biofilm was quantified by measuring the OD₆₂₀ of dissolved CV using a plate reader. Biofilm formation (OD₆₂₀ of CV) was normalized by cell growth (OD₆₂₀) and reported as the relative biofilm formation (OD₆₂₀ of CV/OD₆₂₀ of the culture).

Results:

Biofilm staining with CV at 0.1% revealed high intensity of dying in B24; however, B2538 and DSM 8609 showed very low intensity, both similar to the negative control (medium without the microorganism). The relative biofilm formation was the following: B24, 0.36; B2538, 0.05; and DSM 8609, 0.00. Therefore, B24 had higher biofilm formation than the other tested bacterial strains.

1.5: pH Conditions of Growth

Materials and Methods

pH range of growth at 28° C. of strain B24 was determined in Nutritive Agar (NA) (Scharlab—UNE-EN 12780, EN ISO 16266) plates at different pH. For the experiment, B24 was first grown overnight in LB (5 g/L Yeast Extract; 10 g/L Tryptone; 10 g/L NaCl). After that, B24 was streaked on NA plates at different pHs: 4, 5, 6, 7, 8, 9 and 10. The plates were read after 24h and 4 days of incubation at 28° C. and the bacterial cell growth was evaluated visually.

Results:

The M. esteraromaticum strain B24 grew in pHs: 5, 6, 7, 8, 9 and 10.

1.6: Supernatant of B24 Strain

Materials and Methods

Culture Conditions:

M. esteraromaticum strain B24 was grown in liquid culture medium TSB in 14 L bioreactor at 30° C., 200-450 rpm for 18 hours. For comparison purposes, the M. esteraromaticum collection strain DSM 8609 was used. B24 and DSM 8609 strains were grown in 125 ml of half-strength TSB medium in 250 ml-flask. Culture conditions were at 28° C. 200 rpm for 2 days. After incubation, cell-free culture supernatants (CS) were obtained by centrifugation and filtration through a 0.22 μm pore size filter. To confirm that the supernatants were free of bacterial cells, aliquots of 100 μl were platted in nutritive agar (AN) and absence of growth was confirmed after incubation at 26° C. for 2 days.

In vitro nematicidal activity against root knot nematode Meloidogyne:

The in vitro nematicidal activity of CS samples on nematode eggs was evaluated in hatching chambers (Nunclon™ Surface multiwall plates, Nunc, Denmark) in which 2 g of previously sterilized sand was added. The substrate was then inoculated with an aqueous suspension containing 550 eggs per well (mixture of 50% M. javanica and 50% M. incognita, origin Viladecans, Spain). The hatching chamber was incubated at 26° C. for 2 weeks and readings were done at 7 and 14 days after applying CS sample to determine the hatching percentage. Four repetitions of each treatment and a control with sterile distilled water (to establish a reference to compare the results to the normal hatching percentage) were included. The follow-up of nematode eggs hatching was carried out at 7 and 14 days.

Characterization of Cell-Free Culture Supernatants (CS) by HPLC Analysis:

CS samples from B24 and DSM 8609 grown in TSB medium were analyzed by HPLC (eAlliance system, Waters, Milford, Mass.). Chromatograms were performed using an XBridge C18 reversed-phase column (5 μm 4.6×150 mm, Waters) eluted at 1 ml/min with a mobile phase mixture of solvents A [H₂O+0.05% trifluoracetic acid (TFA)] and B (CH3CN+0.05% TFA). The elution gradient was 0-10% B 10 min, 10-40% B 10 min, 40-100% B 3 min, 100% B 2 min, 0% B 5 min. Produced metabolites were detected by UV spectroscopy with a photodiode array detector and chromatograms were obtained by processing at 254 nm.

Comparison of the Cell-Free Culture Supernatant Enzymatic Activities with Other M. esteraromaticum Strain Supernatant:

The enzymatic activities from cell-free culture supernatants from the B24 M. esteraromaticum strain and the DSM 8609 type strain were evaluated using the API-ZYM® method, as described previously in point 1.2. The culture of the strains and the obtention of the cell-free culture supernatants were performed as described previously herein.

Results:

Two weeks after applying the different treatments, 40.93% of the untreated eggs (control) and 39.22% of the treated eggs with TSB hatched. By contrast, only 29.74% and 27.25% of eggs hatched after treatment with CS of B24 grown in bioreactor and flask, respectively. Therefore, the efficacy of CS of B24 in reducing hatching with respect to the control was around 30% under in vitro conditions.

Root knot nematode eggs treated with CS produced by B24 showed a significant reduction on egg hatching with respect to the control with distilled water or TSB medium. The percentage of efficacy at 14 days after treatment, corrected with respect to the control (distilled water) was: 27.34% efficacy of CS from B24 obtained in bioreactor, in comparison with 4.17% efficacy of TBS medium; and 33.42% efficacy of CS from B24 (flask) compared with 3.46% efficacy of CS from DSM 8609 (flask). The efficacy of CS of the collection strain DSM 8609 grown at the same conditions of B24 was therefore lower. Therefore, the supernatant of the M. esteraromaticum strain B24 had nematicidal effect.

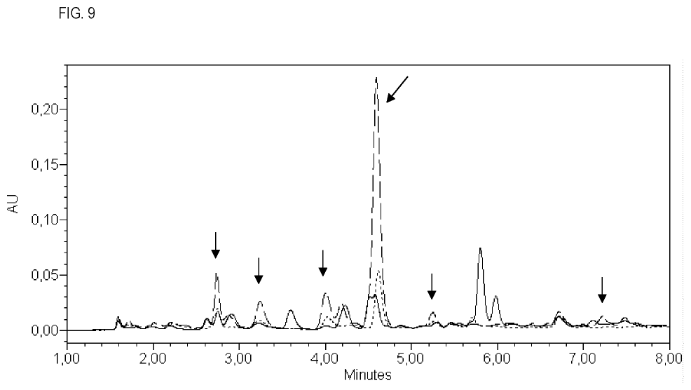

The different chromatographic profiles of B24 and DSM 8609 suggested that each strain produced distinct metabolites (see FIG. 9).

According to API-ZYM®, the CS produced by B24 contained N-acetyl-β-glucosaminidase, leucine arylamidase, α-glucosidase and α-mannosidase. The CS produced by B24 did not contained Alkaline phosphatase, Esterase (C 4), Esterase Lipase (C 8), Lipase (C 14), Valine arylamidase, Cystine arylamidase, Trypsin, α-chymotrypsin, Acid phosphatase, Naphtol-AS-BI-phosphohydrolase, α-galactosidase, β galactosidase, β-glucuronidase, β-glucosidase, nor α-fucosidase.

B24 produced N-acetyl-β-glucosaminidase when it was grown in TSB medium, whereas in solid medium (AN), B24 did not produced this enzyme (as seen in point 1.2).

On the other hand, CS produced by DSM 8609 only showed leucine arylamidase activity. The CS produced by DSM 8609 did not contained N-acetyl-β-glucosaminidase, α-glucosidase and α-mannosidase, Alkaline phosphatase, Esterase (C 4), Esterase Lipase (C 8), Lipase (C 14), Valine arylamidase, Cystine arylamidase, Trypsin, α-chymotrypsin, Acid phosphatase, Naphtol-AS-BI-phosphohydrolase, α-galactosidase, β galactosidase, β-glucuronidase, β-glucosidase, nor α-fucosidase.

According to these results, it was demonstrated that B24 and DSM 8609 produced and liberated different metabolites to the medium. It was demonstrated that the supernatants of both strains were different.

1.7: Genetic Comparison with Other Microbacterium Strains

Materials and Methods

The genome of the B24 strain of the present invention was compared to other Microbacterium species and M. esteraromaticum strains using ANIm described in Richter M, et al. JSpeciesWS: a web server for prokaryotic species circumscription based on pairwise genome comparison. Bioinformatics. 2015 Nov. 16. pii: btv681. Among the Microbacterium species, a comparison was performed using certain strains of the DMSZ German Collection of Microorganisms and Cell Cultures, such as, M. arborescens DSM 20754 [T], M. azadirachtae DSM 23848 [T], M. foliorum DSM 12966 [T], and M. ginsengisoli DSM 18659 [T]. The comparison with other M. esteraromaticum strains was performed using MM1 (GenBank accession number PDVO02) and B Mb 05.01 (GenBank accession number FUKO01) M. esteraromaticum strains.

Results

The ANIm values obtained within the Microbacterium genera was of around 84, a relatively low value within the same genera according to Richter et al. (2015)

In relation to the M. esteraromaticum strains, the ANIm value of the B24 and MM1 was 87.71, the ANIm value of the B24 and B Mb 05.01 was 84.72, and the ANIm value of MM1 and B Mb 05.01 was 84.80.

### Example 2: In Vitro Nematicidal Activity of Microbacterium esteraromaticum Strain B24 Against Eggs of the RKNs

The in vitro nematicidal ability of bacterium M. esteraromaticum strain B24 on a mixture of RKN eggs was evaluated in comparison with a reference chemical (examples 2.1 and 2.2) or in comparison with other M. esteraromaticum strains (example 2.3) (one from the DSMZ collection and another one from the collection of Futureco Bioscience SA).

Materials and Methods

The tests were done in the dark to mimic soil conditions in an incubator (INCUBIG 288L 2000237, JP Selecta, Spain). The incubation temperature was 26° C.

The in vitro nematicidal ability of bacterium M. esteraromaticum strain B24 on a mixture of RKN eggs (M. javanica and M. incognita) (which was obtained from a tomato plant culture) was evaluated in hatching chambers (Nunclon™ Surface multiwell plates, Nunc, Denmark) in which 2 g of previously sterilized sand was added. The substrate was then inoculated with an aqueous suspension containing eggs of the previously mentioned RKNs: 120 eggs in example 2.1; 125 eggs in example 2.2; and 400 eggs in example 2.3. Immediately after, 200 μl of the biological control agent M. esteraromaticum strain B24 was applied at 8.0×10⁸ CFU/mL in example 2.1, at 3×10⁷ CFU/mL in example 2.2 and at 5.20×10⁷ CFU/mL in example 2.3.

In examples 2.1 and 2.2, a control with a reference chemical at the commercially recommended dose (Nemacur® 0.0125%, active ingredient: Fenamiphos 24%, Bayer) and a control with sterile distilled water (to establish a reference to compare the results to the normal hatching percentage) were included.

In example 2.3, comparison was made with two other M. esteraromaticum strains: M. esteraromaticum DSMZ 8609 (applied at 2.03×10⁷ CFU/mL) and M. esteraromaticum strain B2538 (Futureco Bioscience Microorganisms Collection) (applied at 6.20×10⁷ CFU/mL).

The hatching chamber was incubated at 26° C. for 3 weeks and periodic readings were done at 7, 14 and 21 days (in examples 2.1 and 2.2) or at 7 and 14 days (in example 2.3) after applying the biological control agent to determine the hatching percentage (the counts were performed by means of a Hawksley® chamber; microscope OPTECH BIOSTAR B4, Optech Optical Technology, Germany). Six repetitions of each treatment were performed.

Results:

### Example 2.1

M. javanica and M. incognita eggs treated with M. esteraromaticum strain B24 showed a significant reduction on egg hatching with respect to the control (FIG. 1).

Three weeks after applying the different treatments, 27.38% of the untreated eggs (control) hatched, whereas 5.37% of those treated with M. esteraromaticum strain B24 hatched and 2.50% of those treated with Nemacur® (reference chemical) hatched. At day 21 after treatment M. esteraromaticum strain B24 reached 80.36% of efficacy in reducing hatching with respect to the control under in vitro conditions. The efficacy in reducing hatching was similar (there were no statistically significant differences) to the reference chemical (Nemacur®), which had an efficacy in reducing hatching of 90.86%.

### Example 2.2

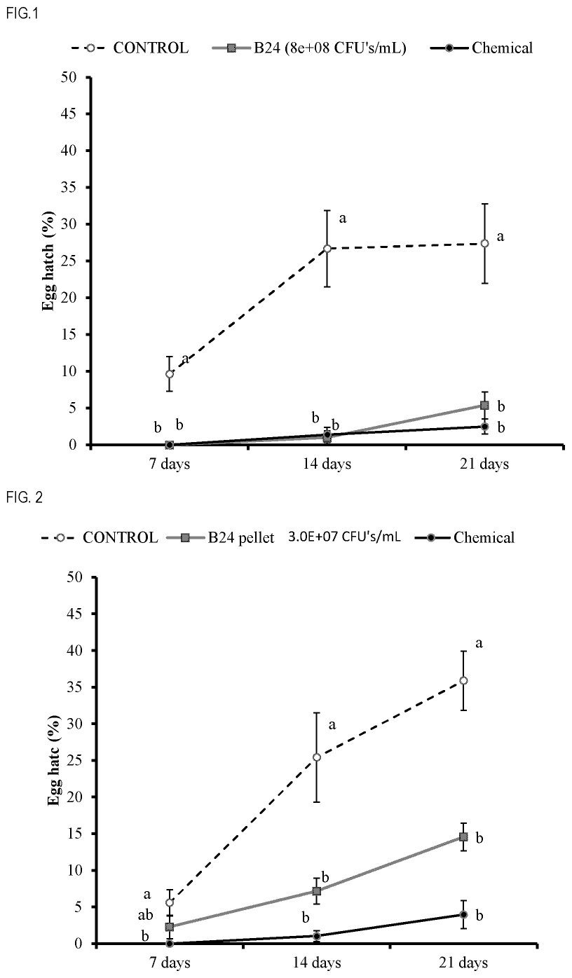

M. javanica and M. incognita eggs treated with M. esteraromaticum strain B24 showed a significant reduction on hatching with respect to the control (FIG. 2).

Three weeks after applying the different treatments, 35.89% of the untreated eggs (control) hatched, whereas 14.57% of those treated with M. esteraromaticum strain B24 hatched and 3.99% of those treated with Nemacur® (reference chemical) hatched. Therefore, M. esteraromaticum strain B24 reached 59.40% of efficacy in reducing hatching with respect to the control under in vitro conditions, whereas in the reference chemical, efficacy in reducing hatching was 88.89%, i.e., higher than the reduction reached by the microorganism object of the present invention (M. esteraromaticum strain B24) but no statistical differences were observed between Nemacur® and M. esteraromaticum strain B24 treatments.

### Example 2.3

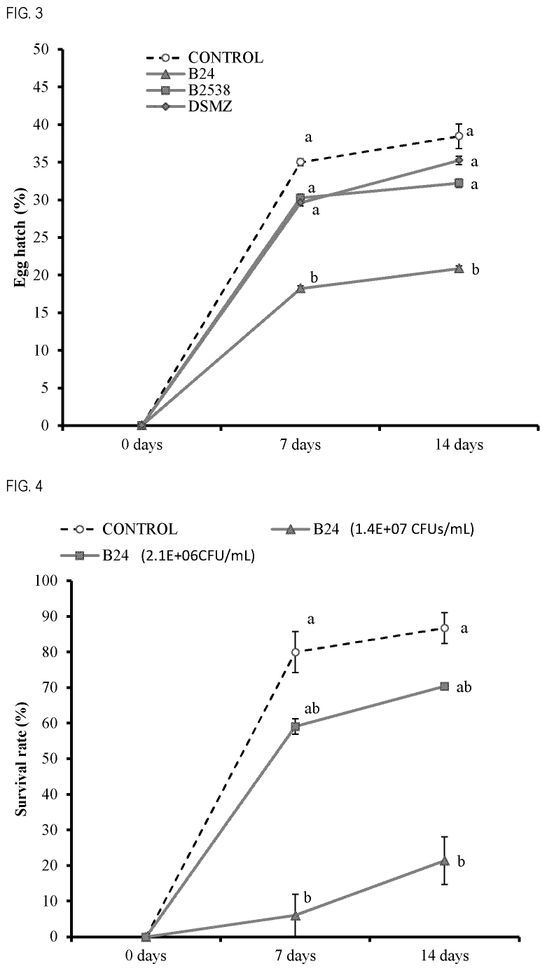

M. javanica and M. incognita eggs treated with M. esteraromaticum strain B24 showed a significant reduction on hatching with respect to the control, the strain B2538 and the reference strain DSMZ 8609 (FIG. 3).

Two weeks after applying the different treatments, 38.46% of the untreated eggs (control) hatched, whereas 20.89% of those treated with M. esteraromaticum strain B24, 35.26% of those treated with M. esteraromaticum strain DSMZ 8609 and 32.23% of those treated with M. esteraromaticum strain B2538 hatched. Therefore, M. esteraromaticum strain B24 reached 45.69% of efficacy in reducing hatching with respect to the control under in vitro conditions, whereas the reference strain (DSMZ 8609) presented 8.32% of efficacy and the strain B2538 showed 16.20% of efficacy.

### Example 3: In Vitro Nematicidal Activity of Microbacterium esteraromaticum Strain B24 Against Juveniles (J2) of RKNs

The in vitro nematicidal ability of bacterium M. esteraromaticum strain B24 was evaluated against a mixture of the RKN M. javanica and M. incognita juveniles (J2).

Materials and Methods:

The tests were done in the dark to mimic soil conditions in an incubator (INCUBIG 288L 2000237, JP Selecta, Spain). The incubation temperature was 26° C. The mixture of RKN M. javanica and M. incognita juveniles (infective juveniles stage, J2) proceeded form a culture in a tomato plant, and they were placed in hatching chambers (Nunclon™ Surface multiwell plates, Nunc, Denmark), in which 2 g of previously sterilized soil were added. Two experiments were performed:

### Example 3.1

An aqueous suspension containing a mixture of 225 juvenile forms of M. javanica+M. incognita was delivered, and then 200 μL of the biological control agent, M. esteraromaticum strain B24 was applied at 1.40×10⁷ CFU/mL or at 2.10×10⁶ CFU/mL. Six repetitions of each treatment were performed.

### Example 3.2

An aqueous suspension containing 275 juvenile forms of the mentioned nematode species (a mixture of them) was delivered, and then three different strains of M. esteraromaticum were applied at different doses: M. esteraromaticum strain B24 was applied at 5.20×10⁷ CFU/mL, M. esteraromaticum DSMZ 8609 was applied at 2.03×10⁷ CFU/mL and M. esteraromaticum strain B2538 was applied at 6.20×10⁷ CFU/mL. The experiment design consisted of three repetitions per treatment.

In both cases (examples 3.1 and 3.2), the hatching chamber was incubated at 26° C. for 14 days and counts were performed by means of the Hawksley® chamber after 7 days and 14 days to determine the number of live juveniles (% survival) (microscope OPTECH BIOSTAR B4, Optech Optical Technology, Germany). A negative control with sterile distilled water was included.

Results:

### Example 3.1

RKN juveniles treated with M. esteraromaticum strain B24 showed a significant reduction on the survival of the juveniles with respect the control when it was applied at the highest dose (1.40×10⁷ cfu/mL) (FIG. 4).

Two weeks after applying the different treatments, 86.75% of the juveniles (negative control) survived, whereas 70.46% of those treated with M. esteraromaticum strain B24 applied at 2.10×10⁶ CFU/mL survived and 21.39% of those treated with M. esteraromaticum strain B24 applied at 1.40×10⁷ survived. Therefore, M. esteraromaticum strain B24 reached 75.34% of efficacy in reducing the survival of juveniles with respect to the control under in vitro conditions when it was applied at a concentration of 1.40×10⁷ CFU/mL, whereas when it was applied at a dose of 2.10×10⁶ CFU/mL, efficacy in reducing juveniles was about 18.78%. Therefore, there were statistical differences between the efficacy achieved by the microorganism object of the present invention (M. esteraromaticum strain B24) when it was applied at the highest dose (1.40×10⁷ CFU/mL).

### Example 3.2

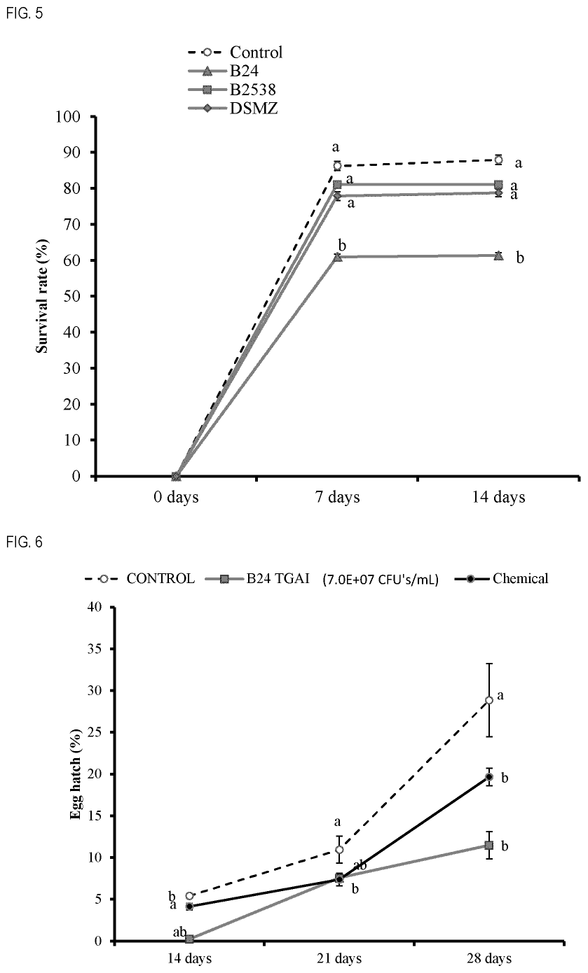

Juveniles treated with M. esteraromaticum strain B24 showed a significant reduction on juveniles survival with respect to strain B2538 and the reference strain DSMZ 8609 (FIG. 5).

Two weeks after applying the different treatments, 87.97% of the untreated juveniles (control) survived, whereas 61.42% of those treated with M. esteraromaticum strain B24 survived, 78.75% of those treated with M. esteraromaticum strain DSMZ 8609 and 81.17% of those treated with M. esteraromaticum strain B2538 survived. Therefore, M. esteraromaticum strain B24 reached 30.19% of efficacy in reducing juvenile survival with respect to the control under in vitro conditions, whereas the reference strain (DSMZ 8609) presented 10.49% of efficacy and the strain B2538 showed 7.73% of efficacy.

### Example 4: In Vitro Nematicidal Activity of Microbacterium esteraromaticum Strain B24 Against Eggs of Potato Cyst Nematodes

The in vitro nematicidal ability of bacterium M. esteraromaticum strain B24 on eggs of Potato Cyst Nematodes (PCNs) Globodera rostochiensis and Globodera paffida was evaluated.

Materials and Methods

The tests were done in the dark to mimic soil conditions in an incubator (INCUBIG 288L 2000237, JP Selecta, Spain). The incubation temperature was 26° C. Hatching chambers (Nunclon™ Surface multiwell plates, Nunc, Denmark) in which 2 g of previously sterilized sand was added were used. Globodera cysts were obtained from an infested soil using a Fenwick Can (flotation method). Cysts were disinfected with 2% bleach for 2 minutes and rinsed with sterile distilled water. Then cysts were then mechanically crushed in order to release Globodera eggs from inside. The in vitro test consisted of three treatments with 4 repetitions each: 1) Control eggs; 2) Eggs treated with M. esteraromaticum strain B24 (at 7×10⁷ CFU/mL)); and 3) Chemical control (Vydate®, Oxamyl 24%, DuPont), applied at the commercial dose 0.2%.

Three hundred and twenty five eggs were placed in each well. Then, 4.5 mL of 10% Potato Root Diffusate (PRD) solution was added per well in order to induce eggs to hatch.

To prepare the PRD, Kennebec variety potato sprouts were seeded in trays containing sterile substrate (peat/vermiculite; 1/1, v/v). Sprouted potatoes were left in a climatic chamber for 3 weeks. Then roots of three-week-old plants were submerged in sterile distilled water for 24 hours at 25° C. After 24 hours, the distilled water that had been in contact with the roots was centrifuged (4000 rpm, 15 minutes), filtered (22 μm) and stored in the freezer until needed.

Once the PRD was added, a solution of M. esteraromaticum strain B24 with a concentration of 7.70×10⁷ CFU/mL was added (200 μL/well). The hatching chambers were maintained at 26° C. throughout the evaluation period. Test readings were taken at 14, 28 and 35 days (using a Hawksley® camber), and 1 mL of new 10% PRD was added every week.

Results

Eggs treated with M. esteraromaticum strain B24 showed a significant reduction on hatching with respect to the control (FIG. 6).

The percentage of efficacy 35 days after treatment with M. esteraromaticum strain B24 was 60.18%, corrected with respect to the control used as reference whereas in the chemical control the percentage of efficacy was 31.90%.

### Example 5: In Vitro Nematicidal Activity of Microbacterium esteraromaticum Strain B24 Against Cysts of PCNs

Materials and Methods

The in vitro nematicidal ability of bacterium M. esteraromaticum strain B24 on cysts of PCN Globodera rostochiensis and Globodera paffida (a mixture of both species 50% each) was evaluated in hatching chambers (Nunclon™ Surface multiwell plates, Nunc, Denmark). The tests were done in the dark in an incubator (INCUBIG 288L 2000237, JP Selecta, Spain).

Globodera cysts were obtained from an infested soil using a Fenwick can (Flotation Method), then they were disinfected as described in example 4, but they were not mechanically crushed. The PRD was obtained as described in the previous bioassay (example 4)

### Example 5.1: A First In Vitro Test Consisted of Three Treatments with 4 Repetitions Each

1) Control cysts—untreated;
2) Control cysts treated with a chemical standard (Vydate®, applied at the commercial dose 0.2%, Oxamyl 24%, DuPont); and
3) Cysts treated with M. esteraromaticum strain B24 as Technical Grade Active Ingredient (TGAI at 7×10⁷ CFU/mL) (TGAI diluted in water).

### Example 5.2: A Second In Vitro Test Consisted of Three Treatments with 4 Repetitions Each

1) Control cysts—untreated;
2) Control cysts treated with a chemical standard (Vydate®, Oxamyl 24%, applied at the commercial dose 0.2%, DuPont); and
3) Cysts treated with M. esteraromaticum strain B24 Formulation OD applied at 1%; Formulation consisted of mixture of soy vegetable oil (Gustave Hess, Germany) 700 g/L+Silica (Silysiamont, Italy) (10 g/L)+C18 ethoxilated fatty acid (Lamberti-Chemical Specialist, Italy) (200 g/L)+polyacrylate crosspolymer-6 (Croda Europe Ltd. United Kingdom) (10 g/L)+TGAI (strain of the invention) (80 g/L). The materials were mixed in a laboratory reactor using a high speed stirrer designed for oils.

The strain of the invention in the TGAI was in a concentration of 1.2-1.3×10¹¹ CFU/g, there for, as used at 8% in the OD formulation, then in said formulation it was at 2.19×10¹⁰ CFU/mL.

Five cysts were placed in each well in both examples. Then, 4.5 mL of 10% PRD solution was added per well in order to induce eggs to hatch. Once the PRD was added, 200 μL per well of a solution of M. esteraromaticum strain B24 with a concentration of 7.70×10⁷ CFU/mL was poured in example 5.1 and 200 μL per well of a solution with M. esteraromaticum strain B24 Formulation OD applied at 1%; was poured in example 5.2. The hatching chambers were maintained at 26° C. throughout the evaluation period. Test readings were taken at 14, 28 and 35 days (using a Hawksley® chamber), and 1 mL of new 10% PRD was added every week.

Results

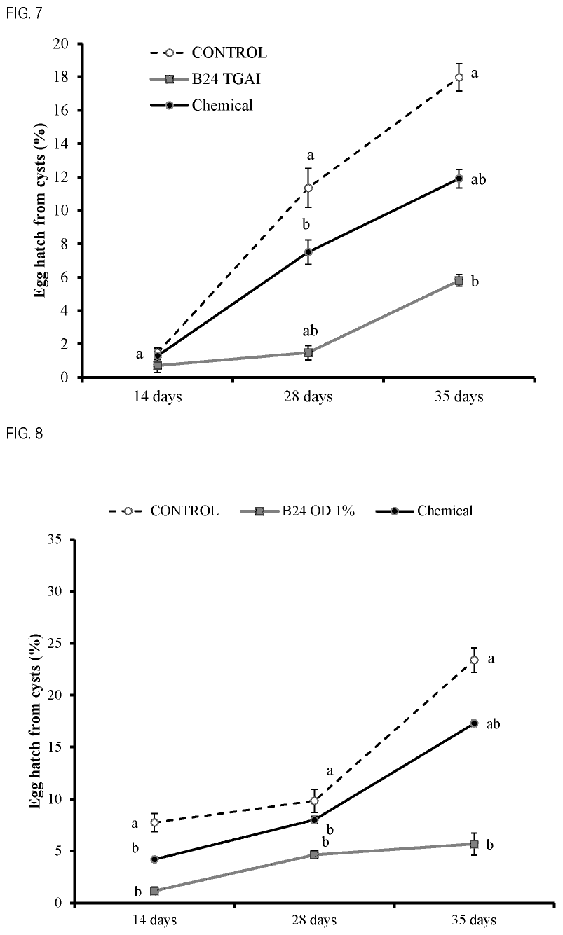

Eggs treated with TGAI M. esteraromaticum strain B24 (Example 5.1.) or Formulation OD 1% of M. esteraromaticum strain B24 (Example 5.2.) showed a significant reduction on hatching with respect the control (FIGS. 7 and 8, respectively).

The percentage of efficacy after 35 days after treatment with TGAI M. esteraromaticum strain B24 was 77.92%, corrected with respect to the control used as reference whereas in the chemical control the percentage of efficacy was 35.72%.

The percentage of efficacy after 35 days after treatment with Formulation OD 1% of M. esteraromaticum strain B24 was 75.72%, corrected with respect to the control used as reference whereas in the chemical control the percentage of efficacy was 26.04%.

### Example 6: In Vivo Nematicidal Activity of Microbacterium esteraromaticum Strain B24 Against RKNs

Three examples were performed using the M. esteraromaticum strain B24 in tomato plants: as “pellet (example 6.1), as TGAI (example 6.2) and as a formulated prototype (example 6.3). The results for all of them are presented at the end of this section. An example using the M. esteraromaticum strain B24 in cucumber plants is disclosed in example 6.4.

Materials and Methods

6.1 In Vivo Nematicidal Activity of a “Pellet” of Microbacterium esteraromaticum Strain B24

The in vivo nematicidal ability of bacterium M. esteraromaticum strain B24 was assessed in two experiments in climatic chamber: against M. javanica+M. incognita. The experimental design was composed by two different treatments: 1) Control with untreated plants; 2) plants treated with a suspension of M. esteraromaticum strain B24 applied as a “pellet”. Each treatment consisted of 3 replicates with 3 plants per replicate.

The pellet was obtained as follows: 1 mL of a frozen sample of the strain B24 (cryovial, −80° C.) was thawed and grown in Tryptic soy broth (TSB) in a 100 mL flask for 3 days approx. at 30° C. It was centrifuged and that pellet that remained (the microorganism as such) was tested in this assay.

### Example 6.1.1

Eighteen tomato seedlings (“Marmande” variety) 3-4 weeks old were transplanted into 1000 cm³ pots filled with a substrate which consisted of a mixture of sand and perlite (3:1; v:v). One day after transplant, all tomato plants were inoculated with a water suspension with root knot nematodes M. javanica+M. incognita (at a dose of 100 infective juveniles per 100 cm³ of substrate) (1000 juveniles/plant were the total addition of both nematodes, they were obtained from a tomato plant). Half of the plants were treated twice with 20 mL of an aqueous solution containing M. esteraromaticum strain B24, firstly 7 DAI (days after nematode inoculation) at a dose of 1.00×10⁷ CFU/mL and secondly 21 DAI at a dose of 1.03×10⁷ CFU/mL (in table 3 below, named as treatments A and B, respectively).

### Example 6.1.2

Eighteen tomato seedlings (“Marmande” variety) 3-4 weeks old were transplanted into 1000 cm³ pots filled with a substrate which consisted of a mixture of sand and perlite (3:1; v:v). Three days before transplant (preventive treatment), one third of the plants were treated with 10 mL of an aqueous solution containing M. esteraromaticum B24 at a dose of 2.13×10⁸ CFU/mL. One day after transplant, all tomato plants were inoculated with a water suspension with root knot nematodes, M. javanica (at a dose of 100 infective juveniles per 100 cm³ of substrate). Then, 20 mL of an aqueous solution containing M. esteraromaticum strain B24 was applied to one third of the plants 7 days after inoculation with the nematodes (7 DAI) at a dose of 4.9×10⁷ CFU/mL. In table 3 below, named as treatments A and B, respectively.

In both experiments (6.1.1 and 6.1.2) the plants were set up in a climatic chamber (temperature: 22±2° C.; relative humidity (RH): 50±10 for 10 weeks since the seedlings were transplanted.

### Example 6.2: In Vivo Nematicidal Activity of Microbacterium esteraromaticum Strain B24 “TGAI” Against RKNs

The in vivo nematicidal ability of bacterium M. esteraromaticum strain B24 was assessed in two experiments in greenhouse trials: example 6.2.1 and example 6.2.2, both against a mixture of M. javanica and M. incognita (obtained from a tomato plant).

The experimental design was composed by two different treatments: 1) Control with untreated plants; 2) Plants treated with a suspension of M. esteraromaticum strain B24 as TGAI (obtained from fermentation in bioreactor) at a dose in average of 7.88×10⁷ cfu/mL in example 6.2.1, and 1.55×10⁸ in average in example 6.2.2. The three treatments consisted of 3 replicates with 3 plants per replicate.

Eighteen tomato seedlings (“Durinta” variety) 3-4 weeks old were transplanted into 3000 cm³ pots filled with a substrate which consisted of a mixture of sand and (3:1; volume:volume). One day after transplant, all tomato plants were inoculated with a water suspension with RKN Meloidogyne javanica+Meloidogyne incognita (at a dose of 100 infective juveniles per 100 cm³ of substrate) (it was a mixture of said nematodes, 50% of Meloidogyne javanica (population of code “AL05” from Futureco Bioscience) plus 50% of Meloidogyne incognita (population of code “AL09” from Futureco Bioscience), the two populations were isolated in Almeria, Spain).

The half of the plants were treated once with 10 mL of an aqueous solution containing M. esteraromaticum strain B24 at a particular dose indicated in the table 1 below three days before transplant (preventive treatment). Then, 20 mL of an aqueous solution containing M. esteraromaticum strain B24 were applied to the same plants 7 days after inoculation with the nematodes (7 DAI), at 21 DAI and at 35 DAI, at the doses indicated in the table 1. Experiment 6.2.1 and 6.2.2 received different doses.

TABLE 1
M. esteraromaticum strain B24 application plant in an in vivo greenhouse trial against M. javanica and
Meloidogyne incognita in tomato plants. In Table 3 below, those treatments are called A, B, C and D.

| | Day 0 | Day 3 | Day 4 | Day 11 | Day 25 | Day 39 |
|---|---|---|---|---|---|---|
| Experiment | 1st Application | Transplant | Nematode | 2^{nd} Application | 3^{nd} Application | 4^{th} Application |
| | (A, preventive) | | inoculation | (B, curative) | (C, curative) | (D, curative) |
| 6.2.1 | 6.2 × 10⁷ | | | 1.50 × 10⁷ | 1.70 × 10⁸ | 1.31 × 10⁸ |
| | CFU/mL | | | CFU/mL | CFU/mL | CFU/mL |
| 6.2.2 | 2.8 × 10⁸ | | | 5.0 × 10⁷ | 1.20 × 10⁸ | 1.68 × 10⁸ |
| | CFU/mL | | | CFU/mL | CFU/mL | CFU/mL |

In both experiments (6.2.1 and 6.2.2) the plants were set up in greenhouse (temperature: 25±2° C.; relative humidity (RH: 50±10) for 9 weeks since the seedlings were transplanted.

### Example 6.3: In Vivo Nematicidal Activity of Microbacterium esteraromaticum Strain B24 “Formulated Prototypes” Against RKNs

The in vivo nematicidal ability of a nematicidal formulation based on the bacterium M. esteraromaticum strain B24 was assessed in two experiments in greenhouse trials in tomato plants (example 6.3.1 and example 6.3.2, both against a mixture of M. javanica and M. incognita (obtained from a tomato plant).

The experimental design was composed by two different treatments: 1) Control with untreated plants; 2) Plants treated with a suspension of bacterium M. esteraromaticum strain B24 applied as a “formulation” at a dose in average 6.43×10⁷ cfu/mL (Formulation-10⁷) in example 6.3.1 or at a dose in average 2.30×10⁷ CFU/mL in example 6.3.2 (Formulation-10⁷). Both treatments consisted of 3 replicates with 3 plants per replicate. Formulation consisted of mixture of: soy vegetable oil (Gustave Hess, Germany) 700 g/L+Silica (Silysiamont, Italy) (10 g/L)+C18 ethoxilated fatty acid (Lamberti-Chemical Specialist, Italy) (200 g/L)+polyacrylate crosspolymer-6 (Croda Europe Ltd. United Kingdom) (10 g/L)+TGAI (strain of the invention) (80 g/L) (the strain of the invention in the TGAI was in a concentration of 1.2-1.3×10¹¹ CFU/g, there for, as used at 8% in the OD formulation, then in said formulation it was at 1×10¹⁰ CFU/mL). The materials were mixed in a laboratory reactor using a high speed stirrer designed for oils.

Eighteen tomato seedlings (“Durinta” variety) 3-4 weeks old were transplanted into 3000 cm³ pots filled with a substrate which consisted of a mixture of sand and perlite (3:1; volume:volume). One day after transplant, all tomato plants were inoculated with a water suspension with root knot nematodes, Meloidogyne javanica+M. incognita (at a dose of approximately 100 infective juveniles (it was a mixture of the two nematodes) per 100 cm³ of substrate). There were two different sets of plants: Half of the plants were untreated (control plants). The other half of the plants was treated once with 20 mL of an aqueous solution containing 1% of a formulation based on M. esteraromaticum strain B24 at a dose indicated in the table 2 below at the day of transplant (preventive treatment). Then, 20 mL of an aqueous solution containing 1% of the formulation based on M. esteraromaticum strain B24 were applied to the same half of the plants 7 days after inoculation with the nematodes (7 DAI), at 21 DAI and at 35 DAI, at the doses indicated in the table 2 below for each experiment.

TABLE 2
Formulation based on M. esteraromaticum strain B24 application
plant in vivo greenhouse trial against RKN in tomato plants.
In Table 4 below, those treatments are called A, B, C and D.

| | Day 1 | Day 2 | Day 9 | Day 23 | Day 37 |
|---|---|---|---|---|---|
| Example | Transplant + | Nematode | 2^{nd} | 3^{nd} Application | 4^{th} Application |
| | 1st Application | inoculation | Application | (C, curative) | (D, curative) |
| | (A, preventive) | | (B, curative) | | |
| 6.3.1 | 2.50 × 10⁸ | | 2.00 × 10⁵ | 1.90 × 10⁶ | 5.20 × 10⁶ |
| | CFU/mL | | CFU/mL | CFU/mL | CFU/mL |
| 6.3.2 | 2.30 × 10⁷ | | 2.00 × 10⁷ | 2.40 × 10⁷ | 2.50 × 10⁷ |
| | CFU/mL | | CFU/mL | CFU/mL | CFU/mL |

In both experiments (6.3.1 and 6.3.2) the plants were set up in greenhouse (temperature: 25±2° C.; relative humidity (RH): 50±10) for 9 weeks since the seedlings were transplanted.

For all experiments (experiments 6.1, 6.2 and 6.3), at the end of the bioassay the total number of eggs per plant and eggs per gram of root were determined in each treatment: they were extracted from the radicular system by mechanical disruption and passed through different sieves from 200 mesh (75 μm) to 500 mesh (25 μm). Eggs were visualized in Hawksley® chamber; microscope OPTECH BIOSTAR B4, Optech Optical Technology, Germany.

Pf in means of number of eggs/plant and nematode Reproduction (eggs/gram of fresh root) was assessed in all treatments 10 or 9 weeks after transplant (10 weeks in the case of the “pellet” and 9 weeks in the case of the TGAI and OD formulation).

In all cases after the treatments performed, data were subjected to analysis of variance (ANOVA) using R software (R Core Team, 2013, Austria). The significant differences among treatments were determined by the Least Significant Difference (LSD) test at P≤0.05.

Results of Examples 6.1 (“Pellet”), 6.2 (TGAI) and 6.3 (Formulated Prototype)

The RKN treated with M. esteraromaticum strain B24 in the three formulations showed a significant reduction on both parameters with respect to the control in all cases (see table 3 below). The efficacy on Reproduction was more similar between treatments as the strain was given in every case per gram of root, whereas the efficacy on Final Population was measured by plant (where differences between development of root system can make differences between repetitions). The results obtained in this assay showed that a formulation based on M. esteraromaticum strain B24 applied as a pellet, as a TGAI or at a 1% OD dose had nematicidal activity against RKN.

TABLE 3
Efficacy (%) of M. esteraromaticum strain B24 (applied as a •pellet”, “TGAI”
or “Formulation” against RKN in tomato plants in greenhouse
corrected with respect to the control used as a reference.

| | | Efficacy (%) on | Efficacy (%) on |
|---|---|---|---|
| | | Final Population | Reproduction |
| Treatments | CFU/mL | (P_{f})(eggs/plant) | (eggs/g root) |
| M. esteraromaticum strain B24 | A: 1.00 × 10⁷ | 72.50 | 68.53 |
| “pellet” (Example 6.1.1.) | B: 1.03 × 10⁷ | | |
| M. esteraromaticum strain B24 | A: 2.13 × 10⁸ | 39.04 | 54.50 |
| “pellet” (Example 6.1.2.) | B: 4.90 × 10⁷ | | |
| M. esteraromaticum strain B24 “TGAI” | A: 6.20 × 10⁷ | 34.77 | 56.30 |
| (Example 6.2.1.) | B: 1.50 × 10⁷ | | |
| | C: 1.70 × 10⁸ | | |
| | D: 1.31 × 10⁸ | | |
| M. esteraromaticum strain B24 “TGAI” | A: 2.80 × 10⁸ | 46.04 | 46.89 |
| (Example 6.2.2.) | B: 5.00 × 10⁷ | | |
| | C: 1.20 × 10⁸ | | |
| | D: 1.68 × 10⁸ | | |
| M. esteraromaticum strain B24 - | A: 2.50 × 10⁸ | 51.28 | 42.46 |
| Formulation (Example 6.3.1) | B: 2.00 × 10⁵ | | |
| | C: 1.90 × 10⁶ | | |
| | D: 5.20 × 10⁶ | | |
| M. esteraromaticum strain B24 - | A: 2.30 × 10⁷ | 57.59 | 56.83 |
| Formulation (Example 6.3.2.) | B: 2.00 × 10⁷ | | |
| | C: 2.40 × 10⁷ | | |
| | D: 2.50 × 10⁷ | | |

### Example 6.4: In Vivo Nematicidal Activity of Microbacterium esteraromaticum Strain B24 Against RKNs in Cucumber Plants

Materials and Methods

The in vivo nematicidal ability of a nematicidal formulation based on the bacterium M. esteraromaticum in cucumber plants was assessed. Similar material and methods as the ones used in examples 6.3 were used, with the exception that cucumber of “Dasher” variety were used, 6 plants per treatment were used (2 plants per block and 3 replicates) and at the doses indicated in the table 4 below. Chemical standard SONDAE (active ingredient Oxamyl 10%, SAPEC AGRO) was used (see table 5 below).

TABLE 4
Formulation based on M. esteraromaticum strain B24 application plant in vivo greenhouse trial
against RKN in cucumber plants. In table 5 below, those treatments are called A, B, C and D.

| | Day 1 | Day 2 | Day 9 | Day 23 | Day 37 |
|---|---|---|---|---|---|
| Treatment | Transplant + 1st | Nematode | 2^{nd} Application | 3^{nd} Application | 4^{th} Application |
| | Application | inoculation | (B curative) | (C curative) | (D curative) |
| | (A preventive) | | | | |
| M. esteraromaticum | 7.50 10⁷ | | 7.50 10⁷ | 7.80 10⁷ | 2.00 10⁷ |
| strain B24 “TGAI” | CFU/mL | | CFU/mL | CFU/mL | CFU/mL |
| (10⁸ CFU/mL.) | | | | | |
| M. esteraromaticum | 3.8 10⁸ | | 3.0 10⁸ | | |
| strain B24 “OD” 2% | CFU/mL | | CFU/mL | | |
| M. esteraromaticum | 2.53 10⁸ | | 2.19 10⁸ | 1.50 10⁸ | 2.00 10⁸ |
| strain B24 “OD” 1% | CFU/mL | | CFU/mL | CFU/mL | CFU/mL |
| M. esteraromaticum | 1.46 10⁸ | | 1.04 10⁸ | 9.70 10⁷ | 1.04 10⁸ |
| strain B24 “OD” 0.5% | CFU/mL | | CFU/mL | CFU/mL | CFU/mL |

Results

In vivo experiments performed in cucumber plants showed good efficacy on final population and efficacy on Reproduction, the RKN treated with M. esteraromaticum strain B24 showed a significant reduction on both parameters with respect to the control (see table 5 below).

TABLE 5
Efficacy (%) of M. esteraromaticum strain B24 (applied as “TGAI”
or “Formulation”) against RKN in cucumber var. “Dasher”
plants in greenhouse corrected with respect to the control used
as a reference. ANOVA, LSD, P < 0.05.

| | | Efficacy (%) on | Efficacy (%) on |
|---|---|---|---|
| | | Final Population | Reproduction |
| Treatments | CFU/mL | (P_{f})(eggs/plant) | (eggs/g root) |
| M. esteraromaticum strain B24 | A: 7.50 10⁷ | 73.96 a | 76.92 a |
| “TGAI” (10⁸ CFU/mL.) | B: 7.50 10⁷ | | |
| | C: 7.80 10⁷ | | |
| | D: 2.00 10⁷ | | |
| M. esteraromaticum strain B24 | A: 3.8.13 × 10⁸ | 68.48 ab | 71.98 a |
| “OD” 2% | B: 3.0 × 10⁸ | | |
| M. esteraromaticum strain B24 | A: 2.53 × 10⁸ | 68.30 ab | 73.78 a |
| “OD” 1% | B: 2.19 × 10⁸ | | |
| | C: 1.50 × 10⁸ | | |
| | D: 2.00 × 10⁸ | | |
| M. esteraromaticum strain B24 | A: 1.46 × 10⁸ | 58.13 bc | 64.24 b |
| “OD” 0.5% | B: 1.04 × 10⁸ | | |
| | C: 9.70 × 10⁷ | | |
| | D: 1.04 × 10⁸ | | |
| Chemical standard (SONDAE, | B: 0.75% | 44.63 c | 46.21 b |
| active ingredient: Oxamyl 10%) | | | |

### Example 7: Evaluation of Oral Toxicity of M. esteraromaticum Strain B24

To determine whether the strain M. esteraromaticum B24 was safe, its oral toxicity in mice was tested.

Materials and Methods:

Three male and three female Balb/C mice of 4-5 weeks old (QC'd albino pathogen free mice; Envigo) were used. Animals fasted overnight before dosing. The day before the experiment, the body weight of the animals was recorded and feces samples were collected from individual animals. Each animal received one oral single dose of the M. esteraromaticum B24 strain: 10⁹ CFU B24 TGAI (vehicle 200 μl of sterile H₂O) (prepared as described previously in example 5.1). Mice that received sterile water (vehicle control) were also maintained throughout the study. Mice were housed under controlled environment and monitored for 21 days and the observations were recorded. Daily, once, the following was observed for each animal: skin and fur, eyes and mucous membranes, respiratory system, circulatory system, autonomic and central nervous system, somatomotor activity, behavior pattern, observation of tremors, convulsions, diarrhea, lethargy, salivation, and coma if any. Weights of individual animals checked prior and post gavage once per week. Necropsy was performed at the end of 21 day post oral gavage for assessment of presence or absence of the bacterial strain in the tissues. Tissue collection and blood culture was done aseptically to avoid any cross contamination.

Necropsy: the kidney, brain, liver, lung, spleen, blood and gut lymph node was collected from each animal and immediately kept in dry ice and stored at −20° C. until further processing. RNA was extracted from the above tissues and RT-PCR was performed to determine presence of B24 TGAI (16S rRNA) using the specific primers: “M estera F1”, (SEQ ID NO: 4); “M estera R1”, (SEQ ID NO: 5); “M estera F2”, (SEQ ID NO: 6); and “M estera R2” (SEQ ID NO: 7) (the two primer pairs were used in order to be sure of the specificity of the amplification; R, reverse primer; F, forward primer).

Blood Processing: at necropsy, aseptic whole blood samples were collected from each animal. Immediately following collection, 100 μl of each blood sample was placed and spread on agar plate and maintained at 28° C. for 120 hours. The plates were analyzed at regular intervals for assessment of any bacterial growth.

Clearance of the Microbial Pest Control Agent (MPCA): feces collection on dosing and on day 7, 14 and 21 days were kept at 4° C. for further RNA extraction and RT-PCR analysis.

Blood/Bacterial culture media: Meat extract 1 g, Yeast Extract 2 g, Peptone 5 g, NaCl 5 g and Agar 15 g were added (for 1000 ml) and sterilized for 121° C. for 20 min and thereafter was poured into plates and used for the blood culture and B24 TGAI CFU/g determination. RNA extraction protocol: 50 mg of tissue was used for RNA extraction following manufacturer's instructions (RNA Isolation kit Zymo Research, Cat No R1050).

Spike control: 100 μl of 10⁹ CFU of B24 TGAI was spiked with 50 mg of brain and liver tissue and total RNA was extracted by using the Quick RNA kit total RNA extraction kit according to manufacturer's instructions (Zymo Research) with addition of Proteinase K and Lysozyme digestion step—included in the aforementioned kit—for isolating total RNA including bacterial RNA.

Positive control: 0.2 g of B24 TGAI diluted serially for 7 times in sterile water and plated (100 μl) and re-confirmed the CFU/g sample. The bacterial colonies grown on agar plates were collected and directly used for total RNA extraction and as a positive control for the study.

cDNA synthesis: Total RNA from tissues and feces were measured in a micro plate reader (Biotek). 2 μg of total RNA (1 pg in case of feces samples) was used for cDNA synthesis by using Superscript IV 1st strand synthesis kit (Life technologies, Cat No: 18091050). Appropriate positive (B24 TGAI) and negative controls (H₂O) were also maintained.

RT-PCR assay: 20 μl total of the cDNA mixture was diluted 1:10 fold (tissue samples) and 1:5 fold (feces samples cDNA) and RT-PCR was performed using PowerUp SYBR™ Green Master Mix according to manufacturer's instructions (Life Technologies, Cat No A25776) with an Applied Biosystems StepOne™ real time PCR device. PCR reactions consisted of 5 μL PowerUp™ SYBR™ Green Master Mix, 0.5 μL each specific primer (F1 and R1, or F2 and R2), and 10 ng cDNA as template, in a total reaction volume of 20 μL. The thermal cycling conditions were as follows: 2 min at 50° C., 2 min at 95° C., and 40 cycles of 15 s at 95° C., 15 s at 60° C., and 1 min at 72° C.

Results:

Body weight from the control and M. esteraromaticum B24 gavaged animal were not significantly different. Blood culture plates incubated for 120h at 28 degree had no visible growth and was concluded negative. Clinical symptoms monitored did not reveal adverse changes. RT-PCR data revealed that the 16srDNA specific primers were not amplified in any of the tissue or feces samples from M. esteraromaticum B24 gavaged animals (Average Ct values 33). The M. esteraromaticum B24 TGAI spiked tissue samples were amplified using the M esteraromaticum specific primers along with the positive control (average spiked Ct value 12 for brain and 17 for liver). Negative controls (water gavaged) had no amplification in RT-PCR (Average Ct value 30). Positive control 16s rDNA primer amplified at cycle 6 (Ct value 6). The data revealed absence of M. esteraromaticum B24 in any of the samples screened using the specific RT-PCR primers.

Animals had no visible clinical adverse effects and no infectivity or pathogenicity was found. M. esteraromaticum B24 strain gavaged animals had no visible clinical adverse effects and the bacterial strain was not detected in the blood, feces or other organs examined.

### REFERENCES CITED IN THE APPLICATION

Sturz A V and Kimpinski J “Endoroot bacteria derived from marigolds (Tagetes spp.) can decrease soil population densities of root-lesion nematodes in the potato root zone” 2004 Plan and Soil 262:241-249.
Sambrook J and Russell D W “Molecular Cloning: A Laboratory Manual” Chapter 13 “Mutagenesis” Cold Spring Harbor, 3rd Ed, 2001.
Schwyn B and Neilands J B “Universal chemical assay for the detection and determination of siderophores.” 1987 Anal Biochem 160(1): 47-56.
Altschul et al., “Basic local alignment search tool”, 1990, J. Mol. Biol, v. 215, pages 403-410.
Higgins et al., “CLUSTAL V: improved software for multiple sequence alignment”, 1992, CABIOS, 8(2), pages 189-191.
Richter M, et al. JSpeciesWS: a web server for prokaryotic species circumscription based on pairwise genome comparison. Bioinformatics. 2015 Nov. 16. pii: btv681.
Aubert et al., “A Markerless Deletion Method for Genetic Manipulation of Burkholderia cenocepacia and Other Multidrug-Resistant Gram-Negative Bacteria” Methods Mol Biol 2014; 1197:311-27.

For reasons of completeness, various aspects of the invention are set out in the following numbered clauses:

Clause 1. A strain of Microbacterium esteraromaticum deposited at the “Colección Espanola de Cultivos Tipo” (CECT) under the accession number CECT9167, or a mutant thereof, wherein said mutant is obtained using the CECT9167 of Microbacterium esteraromaticum as starting material and maintains the nematicidal effect of CECT9167.

Clause 2. A bacterial culture comprising the strain or the mutant thereof of clause 1, preferably the bacterial culture is an inoculation product.

Clause 3. A composition comprising an effective amount of the strain CECT9167 of Microbacterium esteraromaticum or the mutant thereof of clause 1 or the bacterial culture of clause 2, and one or more agriculturally acceptable compounds.

Clause 4. The composition of clause 3 wherein the strain is present at a concentration from 10⁵ CFU/ml to 10¹² CFU/ml.

Clause 5. The composition of any one of clauses 3 or 4 wherein the agriculturally acceptable compound is selected from the group consisting of: plant strengtheners, nutrients, wetting agents, compounds that improve adherence, buffering compounds, stabilizers, antioxidants, osmotic protectors and sunscreens.

Clause 6. The composition of any one of clauses 3 to 5, which comprises at least one additional pesticide.

Clause 7. The composition of any one of clauses 3 to 6 wherein the composition comprises an oily ingredient, preferably a vegetable oil, preferably soy oil.

Clause 8. The composition of any one of clauses 3 to 7 wherein the composition comprises soy oil, C18 ethoxilated fatty acid, silica and polyacrylate crosspolymer.

Clause 9. A method to obtain a mutant of the strain of CECT9167 of M. esteraromaticum which maintains the nematicidal effect of CECT9167, comprising the step of subjecting the strain CECT9167 to a DNA recombinant technique, preferably mutagenesis.

Clause 10. A process for obtaining a viable cell suspension derived from the strain CECT9167 of Microbacterium esteraromaticum or a mutant thereof of clause 1, the process comprising: (i) inoculating the strain in a suitable culture medium, (ii) subjecting the inoculated culture medium of the step (i) to conditions suitable for growth of the strain, and (iii) optionally subjecting the medium resulting from step (ii) to a concentration step.

Clause 11. A supernatant derived from the strain CECT9167 of M. esteraromaticum or a mutant thereof as defined in clause 1, said supernatant being obtainable by a process comprising: (i) inoculating the strain in a suitable culture medium; (ii) subjecting the inoculated culture medium to suitable growth conditions; (iii) separating the cells from the culture medium of step (ii); (iv) collecting the supernatant; and (v) optionally subjecting the supernatant to a concentration step.

Clause 12. A kit comprising an effective amount of the strain CECT9167 of M. esteraromaticum or the mutant thereof of clause 1, the bacterial culture of clause 2, the composition of any one of clauses 3 to 8 or the supernatant of clause 11.

Clause 13. Use of the strain CECT9167 of Microbacterium esteraromaticum or the mutant thereof of clause 1, or of the bacterial culture of clause 2, or of the composition of any one of clauses 3-8, or of the supernatant of clause 11, or of the kit of clause 12 for controlling a nematode infection in a plant.

Clause 14. A method for controlling an infection caused by nematodes in a plant comprising applying to a part of a plant or to the substrate used for growing said plant the strain CECT9167 of Microbacterium esteraromaticum or the mutant thereof of clause 1, or the bacterial culture of clause 2, or the composition of any one of clauses 3-8, or the supernatant of clause 11, or the kit of clause 12.

Clause 15. The use of clause 13 or the method of clause 14, wherein: the nematode is selected from the genus Meloidogyne and Globodera; or alternatively, it is selected from the group consisting of Meloidogyne incognita, Meloidogyne javanica, Globodera rostochiensis and Globodera paffida.

1. A pesticide comprising a Microbacterium esteraromaticum strain deposited at the “Coleccion Española de Cultivos Tipo” (CECT) under the accession number CECT9167, or a mutant thereof, wherein said mutant is obtained using the CECT9167 of Microbacterium esteraromaticum as starting material and maintains the nematicidal effect of CECT9167.

2. The pesticide according to claim 1 wherein the mutant of the strain CECT9167 of Microbacterium esteraromaticum has a genomic sequence identity of at least 99.8% with the strain CECT9167 of Microbacterium esteraromaticum.

3. The pesticide according to claim 1 which comprises an inoculation product comprising the strain according to claim 1.

4. The pesticide according to claim 1, which further comprises one or more agriculturally acceptable compounds.

5. The pesticide according to claim 1 wherein the strain is present at a concentration from 10⁵ CFU/ml to 10¹² CFU/ml.

6. The pesticide according to claim 4 wherein the agriculturally acceptable compound is selected from the group consisting of: plant strengtheners, nutrients, wetting agents, compounds that improve adherence, buffering compounds, stabilizers, antioxidants, osmotic protectors and sunscreens.

7. The pesticide according to claim 4, which comprises at least one additional pesticide.

8. The pesticide according to claim 4 wherein the composition comprises an oily ingredient, preferably a vegetable oil, preferably soy oil.

9. The pesticide according to claim 4 wherein the composition comprises soy oil, C18 ethoxilated fatty acid, silica and polyacrylate crosspolymer.

10-11. (canceled)

12. A process for obtaining a viable cell suspension of the strain CECT9167 of Microbacterium esteraromaticum or a mutant thereof according to claim 1, the process comprising: (i) inoculating the strain in a suitable culture medium, (ii) subjecting the inoculated culture medium of the step (i) to conditions suitable for growth of the strain, and (iii) optionally subjecting the medium resulting from step (ii) to a concentration step.

13. A supernatant of the strain CECT9167 of M. esteraromaticum or a mutant thereof as defined claim 1, said supernatant being obtainable by a process comprising: (i) inoculating the strain in a suitable culture medium; (ii) subjecting the inoculated culture medium to suitable growth conditions; (iii) separating the cells from the culture medium of step (ii); (iv) collecting the supernatant; and (v) optionally subjecting the supernatant to a concentration step.

14-15. (canceled)

16. A method for controlling an infection caused by nematodes in a plant comprising applying to a part of a plant or to the substrate used for growing said plant the pesticide according to claim 1.

17. The method according to claim 16, wherein: the nematode is selected from the genus Meloidogyne and Globodera; or alternatively, it is selected from the group consisting of Meloidogyne incognita, Meloidogyne javanica, Globodera rostochiensis and Globodera pallida.

18. The pesticide according to claim 7, wherein the additional pesticide is selected from the group consisting of another bacterial strain with pesticide properties, a fungicide, a bactericide, an herbicide, an insecticide and a chemical nematicide.

19. The supernatant according to claim 13 wherein the culture rmedium is liquid medium and the supernatant comprises N-acetyl-β-glucosaminidase, leucine arylamidase, α-glucosidase and α-mannosidase; or, alternatively, wherein the culture medium is solid medium and the supernatant comprises leucine arylamidase, α-glucosidase and α-mannosidase.

20. The method of claim 16, wherein the nematode is a root-knot nematode or a cyst nematode.

21. The method of claim 16, wherein the pesticide comprising the strain CECT9167 of M. esteraromaticum, or a mutant thereof as defined in the first aspect of the invention is administered to the plant at a dosage regime of 10⁶-10⁸ CFU/g or 10⁶-10⁸ CFU/mL per day once during several weeks.

22. The method of claim 16, wherein the pesticide is applied to plant nursery trays, during seed treating, seed dressing, seed disinfection, seedling root dipping treatment, planting pit treatment, plant foot treatment, planting row treatment, surface spraying, soil incorporation, drip irrigation, or by application to a water culture medium in hydroponic culture.

23. The method according to claim 16, wherein the part of a plant is the root, stem, leaves or seeds.

24. A method for controlling an infection caused by nematodes in a plant comprising applying to a part of a plant or to the substrate used for growing said plant the supernatant according to claim 13.

25. The method of claim 24 wherein the nematodes are selected from the genus Meloidogyne and Globodera; or alternatively, it is selected from the group consisting of Meloidogyne incognita, Meloidogyne javanica, Globodera rostochiensis and Globodera pallida.


SEQUENCE LISTING (7 sequences)

SEQ ID NO: 1 - DNA, 18 bp, Artificial Sequence
  oligonuclotide for 16S rRNA sequencing
agtttgatcctggctcag

SEQ ID NO: 2 - DNA, 21 bp, Artificial Sequence
  oligonucleotide for 16S rRNA sequencing
acggttaccttgttacgactt

SEQ ID NO: 3 - DNA, 970 bp, Artificial Sequence
  amplicon
tgcaagtcgaacgatgaagcccagcttgctgggtggattagtggcgaacgggtgagtaacacgtgagcaacctgcccctgactctgggataagcgctggaaacggcgtctaatactggatatgtcccgtcaccgcatggtgtgcgggtggaaagatttttcggttggggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcggaagcctgatgcagcaacgccgcgtgagggatgacggccttcgggttgtaaacctcttttagcagggaagaagcgaaagtgacggtacctgcagaaaaagcaccggctaactacgtgccagcagccgcggtaatacgtagggtgcaagcgttatccggaattattgggcgtaaagagctcgtaggcggtctgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtcctttccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatacacgagaacgggccagaaatggtcaactctttggacactcgtgaacag

SEQ ID NO: 4 - DNA, 20 bp, Artificial Sequence
  primer
cctcgttctatgttgccagc

SEQ ID NO: 5 - DNA, 20 bp, Artificial Sequence
  primer reverse
tgcagacctcaatccgaact

SEQ ID NO: 6 - DNA, 20 bp, Artificial Sequence
  Primer forward
ctgggataagcgctggaaac

SEQ ID NO: 7 - DNA, 20 bp, Artificial Sequence
  primer reverse
caatattccccactgctgcc